SEQUENCE LISTING (8 sequences)

SEQ ID NO: 1 - RNA, 21 nt, synthetic construct
  source 1..21: mol_type = other RNA
cagcaatgttctccgtaaatt

SEQ ID NO: 2 - RNA, 21 nt, synthetic construct
  source 1..21: mol_type = other RNA
tggtcgttacaagaggcattt

SEQ ID NO: 3 - RNA, 21 nt, synthetic construct
  source 1..21: mol_type = other RNA
ctgatatgctcctcgactatt

SEQ ID NO: 4 - RNA, 21 nt, synthetic construct
  source 1..21: mol_type = other RNA
tggactatacgaggagctgat

SEQ ID NO: 5 - RNA, 21 nt, synthetic construct
  source 1..21: mol_type = other RNA
gatcatgcgcacagtctattt

SEQ ID NO: 6 - RNA, 21 nt, synthetic construct
  source 1..21: mol_type = other RNA
tgctagtacgcgtgtcagata

SEQ ID NO: 7 - RNA, 21 nt, synthetic construct
  source 1..21: mol_type = other RNA
ctgcactaggctttagaaatt

SEQ ID NO: 8 - RNA, 21 nt, synthetic construct
  source 1..21: mol_type = other RNA
tggacgtgatccgaaatcttt

(19) United States
(12) Patent Application Publication
(10) Pub. No.: US 2026/0308588 A1
(43) Pub. Date: Oct. 8, 2026

(54) METABOLIC TARGET FOR THERAPY OF CHRONIC VASCULAR INFLAMMATION

(71) Applicant: Yale University, New Haven, CT (US)

(72) Inventors: Michael Simons, New Haven, CT (US); Pei Yu Chen, New Haven, CT (US)

(21) Appl. No.: 19/482,829

(22) PCT Filed: May 10, 2024
(86) PCT No.: PCT/US2024/028722

§ 371 Date: Nov. 10, 2025

Related U.S. Application Data
(60) Provisional application No. 63/501,556, filed on May 11, 2023.

(51) Int. Cl. C12N 15/113; A61K 31/711; A61P 9/10

(52) U.S. Cl. CPC C12N 15/113; A61K 31/711; A61P 9/10; C12N 2310/14

(57) ABSTRACT

The present invention includes compositions and methods useful for the treatment of chronic vascular inflammation through the inhibition of ACSS2. In various embodiments, the EndMT occurs as a result of chronic vascular inflammation. In various embodiments, the inhibition of ACSS2 opposes pathogenic Transforming Growth Factor-Beta (TGFβ) in the cell.

### CROSS-REFERENCE TO RELATED APPLICATION

The present application is entitled to priority under 35 U.S.C. § 119(e) to U.S. Provisional Patent Application No. 63/501,556, filed May 11, 2023, which is incorporated herein by reference in its entirety.

### STATEMENT REGARDING FEDERALLY SPONSORED RESEARCH OR DEVELOPMENT

This invention was made with government support under HL144939 awarded by National Institutes of Health. The government has certain rights in the invention.

### BACKGROUND OF THE INVENTION

A number of studies have established endothelial-to-mesenchymal transition (EndMT) as the key pathological event responsible for chronic vascular inflammation. EndMT underlies and drivers a wide spectrum of diseases from atherosclerotic cardiovascular disease (myocardial infarction, strokes) to pulmonary hypertension to vascular graft failure to numerous fibrotic states. The key to EndMT development is activation of endothelial TGFβ signaling, which drives the downstream processes responsible for the transition into mesenchymal-like cells. Most current strategies to block or inhibit EndMT involve targeting expression or function of TGFβ and its receptor. However, more complete characterization of the metabolic pathways involved in EndMT could identify more effective targets for blocking this process.

As such a need exists for methods of blocking or reducing cellular signals that lead to EndMT as a treatment for chronic vascular inflammatory diseases. The present invention addresses this need.

### SUMMARY OF THE INVENTION

As described herein, the present disclosure relates to compositions and methods useful for the treatment of chronic vascular inflammation through the inhibition of ACSS2.

In one aspect, the invention includes a method of inhibiting endothelial-mesenchymal transition (EndMT) in a cell comprising contacting the cell with an effective amount of an inhibitor of Acetyl COA Synthetase Short chain family member 2 (ACSS2).

In certain embodiments, the EndMT occurs as a result of chronic vascular inflammation.

In certain embodiments, the inhibition of ACSS2 opposes pathogenic Transforming Growth Factor-Beta (TGFβ) in the cell.

In certain embodiments, inhibition of EndMT is used to treat a disease associated with chronic vascular inflammation.

In certain embodiments, the disease is selected from the group consisting of atherosclerosis, arteriosclerosis, pulmonary hypertension, myocardial infarction, and stroke.

In certain embodiments, the inhibitor of ACSS2 is an inhibitory RNA which reduces the expression of ACSS2.

In certain embodiments, the inhibitory RNA is an siRNA.

In certain embodiments, the siRNA is encoded by a nucleic acid sequence set forth in SEQ ID NOs: 1-8.

In another aspect, the invention includes a pharmaceutical composition comprising:
a. an inhibitor of ACSS2,
b. a lipid nanoparticle, and
c. a pharmaceutically acceptable carrier.

In certain embodiments, the inhibitor of ACSS2 is an inhibitory RNA which reduces the expression of ACSS2.

In certain embodiments, the inhibitory RNA is an siRNA.

In certain embodiments, the siRNA is encoded by the nucleotide sequence set forth in SEQ ID NO:s 1-8.

In another aspect, the invention includes a method of treating a vascular inflammatory disease in a subject in need thereof, the method comprising administering an effective amount of an inhibitor of Acetyl COA Synthetase Short chain family member 2 (ACSS2).

In certain embodiments, the vascular inflammatory disease is a chronic vascular inflammation disease

In certain embodiments, the chronic vascular inflammatory disease is selected from the group consisting of atherosclerosis, myocardial infarction, pulmonary hypertension, and stroke.

In certain embodiments, the vascular inflammatory disease results from a vascular graft failure.

In certain embodiments, the vascular inflammatory disease is associated with a fibrotic state of the vessels.

In certain embodiments, the inhibitor of ACSS2 is an inhibitory RNA that reduces the expression of ACSS2.

In certain embodiments, the inhibitory RNA is an siRNA.

In certain embodiments, the siRNA is encoded by the nucleic acid sequence set forth in SEQ ID NOs. 1-8.

In certain embodiments, wherein the ACSS2 inhibitor is the pharmaceutical composition of any one of the above aspects or embodiments, or any aspect or embodiment disclosed herein.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1F illustrate that TGFβ signaling regulates endothelial cell metabolism by suppressing PDK4 transcription.

FIG. 2A-2D illustrate that TGFβ signaling activates PDH complex by dephosphorylating PDHE1α.

FIG. 3 is a diagram showing the steps of glucose metabolism and enzymes active at each step.

FIGS. 4A-4H illustrate that PDK4 deficiency can induce Endo-MT progression of HUAECs.

FIGS. 5A-5H illustrate that TGFβ2 treatment promotes the production of Acetyl-CoA in artery endothelial cells.

FIG. 6 is a diagram of the mechanism by which TGFβ signaling leads to increased acetyl-CoA production by the PDH complex via inhibition of PDKs.

FIG. 7 is a diagram illustrating the production of acetyl-CoA from glucose or acetate.

FIGS. 8A-8C illustrate that acetate treatment moderately promotes EndMT of arterial endothelial cells.

FIGS. 9A-9F illustrate that ACSS2 deficiency significantly rescues TGFβ induced EndMT.

FIGS. 10A-10L illustrate that reducing ACSS2 expression significantly rescues EndMT induced by the loss of function of PDK4.

FIGS. 11A-11D illustrate that ACSS2 deficiency enhances the phosphorylation of PDHE1α by increasing PDK4 levels.

FIGS. 12A-12E illustrate that the rescue of TGFβ induced EndMT by reduction of ACSS2 expression can be reversed by attenuating PDK4 expression

FIG. 13 is a diagram illustrating the Acetate--ACSS2--Acetyl-CoA axis.

FIGS. 14A-14H illustrate that ACSS2 deficiency excludes the nuclear SMAD2 and inhibits its transcriptional activity by reducing its acetylation.

FIG. 15 is a diagram illustrating the role of TGFβ signaling in driving EndMT and ACSS2 ablation inhibiting EndMT.

FIG. 16 is a diagram illustrating the possible inhibition points of acetyl-CoA production.

FIG. 17 is a series of micrographs demonstrating that ACSS2 expression is higher in human endothelial tissue from patients with severe atherosclerosis as compared to healthy controls.

FIG. 18 illustrates that ACSS2 expression can be successfully targeted by siRNA inhibition.

FIG. 19 illustrates the successful knockdown of ACSS2 expression in human endothelial cells.

FIGS. 20A-20K illustrate the metabolic effects of endothelial TGFβ signaling. FIG. 20A. Representative blots for key glycolytic proteins in HUAECs before and after TGFβ2 stimulation for 7 days. FIG. 20B. Diagram of carbon flux in glycolysis. FIG. 20C-E. Glycolytic activity was measured in HUAECs with or without TGFβ2 stimulation (10 ng/mL) for 7 days. Glycolytic flux was then determined by testing the conversion of glucose, D-[5-3H(N)] to 3H2O (F). 2-deoxyglucose (2-DG) and conversion to 2-DG-6-phosphate (2-DG6P) were measured to show the glucose uptake changes (FIG. 20D). Lactate concentration in the media collected from HUAECs culture under indicated conditions at specified time points normalized by the cell number (FIG. 20E). 5% dialyzed fetal bovine serum (D-FBS) with lower basal lactate was to the medium instead of FBS. FIGS. 20F-20K. Liquid chromatography-mass spectrometry (LC-MS) metabolomics analysis of key metabolites in HUAECs treated with 13C-glucose (U-13C6-glucose, 10 mM) for 24 h after 7 days of TGFβ2 stimulation. Total ion counts of key intermediaries of glycolysis can be found in (I), the change in the ribose phosphate which indicates the level of pentose phosphate pathway is shown in (FIG. 20G), 13C-glucose contribution to biosynthesis of TCA intermediates is shown (FIG. 20H). Changes in the transcript copy number of TCA-related genes from RNA-seq (FIG. 20I). Changes in the oxygen consumption rate (OCR) (FIG. 20J) and extracellular acidification rate (ECAR) (FIG. 20K) in HUAECs during TGFβ driven EndoMT were analyzed. The data in FIGS. 20C-20G, and FIGS. 20I-20K were normalized to those of the non-TGFβ-treated control or scramble siRNA-treated cells and are presented as the mean±SEM from at least three independent experiments. ****p % 0.0001, ***p % 0.001, **p % 0.01, *p % 0.05, NS, not significant.

FIGS. 21A-21T illustrate that TGFβ regulates PDK4 expression and Ac-COA synthesis, and increase in endothelial Ac-COA levels induces EndMT in ACSS2-dependent manner (FIGS. 21A and 21B) After 7 days of TGFβ2 stimulation (10 ng/ml), the levels of phosphorylated PDHE1α and total PDK4 in HUAECs cell lysates (FIG. 21A) and Ac-CoA level in the cytosol of HUAECs (FIG. 21B) were determined. (FIGS. 21C and 21D) Scramble siRNA or PDK4 siRNA were transduced into HUAECs for 7 days, blocking efficiency of PDK4 siRNA, and the levels of phosphorylated PDHE1α were analyzed using 20 mg lysate of HUAECs (C). Cytosolic Ac-CoA levels in 1.5 million HUAECs (FIG. 21D). (FIG. 21E) Representative blots showing the effect of the PDKs inhibitor, dichloroacetate sodium (DCA) on PDHE1αactivity in HUAECs treated with DCA for 3 days. (FIG. 21F) After DCA (10 mM) treatment for 7 days, the cytosolic Ac-CoA level in 1.5 million HUAECs was determined. (FIGS. 21G-I) Chromatin immunoprecipitation (ChIP)-QPCR testing of SMAD-2/3 binding to SBEs in human PDK4 promoter in human umbilical vein endothelial cells (HUVECSs) (FIG. 21G) and HUAECs (FIG. 21J). After 5 days of TGFβ2 stimulation, the direct regulation of PDK4 expression by SMADs was analyzed (FIG. 21I). (FIGS. 21J-21M) Pharmacological inhibition of PDKs and PDK4 knockdown induce EndMT. Cell shape transition of HUAECs after DCA treatment (FIG. 21J) or siRNA-mediated PDK4 deficiency (FIG. 21L) for 7 days. Scale bars, 15 mm. Representative blots showing EndMT markers and EC-specific gene expression in HUAECs treated with DCA (FIG. 21K) or PDK4 siRNA (FIG. 21M) for 7 days. (FIGS. 21N and 21O) In HUAECs, the contributions of ACLY (FIG. 21N) and ACSS2 (FIG. 21O) to Ac-CoA production. (FIG. 21P) Cytosolic Ac-CoA level were measured in HUAECs transduced with Scramble siRNA, Scramble siRNA+TGFβ2, or ACSS2 siRNA+TGFβ2, respectively. (FIGS. 21Q and 21R) Blots showing EndMT markers and EC-specific markers in HUAECs treated with Scramble siRNA, Scramble siRNA+TGFβ2, or ACSS2 siRNA+TGFβ2 (FIG. 21Q)/ACLY siRNA+TGFβ2 (FIG. 21R) separately for 7 days. (FIG. 21S) Changes of EndMT markers and EC-specific markers were determined in HUAECs treated with Adv-CTL, Adv-CTL+TGFβ2, or Adv-ACSS2-HA+TGFβ2 separately for 3 days. (FIG. 21T) Bulk RNA-seq analysis of EndMT markers and EC-specific gene expression in HUAECs under different conditions: Scramble siRNA, Scramble siRNA+TGFβ2, or ACSS2 siRNA+TGFβ2. The data in FIGS. 21B, 21D, 21F, and 21N-P were normalized to those of the non-TGFβ-treated control or scramble siRNA-treated cells and are presented as the mean±SEM from at least three independent experiments. **p % 0.01, *p % 0.05, NS, not significant.

FIGS. 22A-22F illustrate that PDK4, inhibited by canonical TGFβ/RSMADs signaling, plays crucial roles in regulating PDH phosphorylation. FIG. 22A, Representative blots for PDK proteins and PDH phosphatase 1 in HUAECs treated with 7 days of TGFβ2 stimulation (10 ng/ml). FIG. 22B, Representative blots for PDK proteins in HUAECs transduced with Scramble siRNA or PDK4 siRNAs for blocking PDK4. FIGS. 22C-22D EndMT markers, EC markers, and PDH phosphorylation were analyzed in HUAECs transduced with siRNAs for blocking PDK1 or PDK2 separately (FIG. 22C), or for blocking PDK3 or PDK4 separately (FIG. 22D). FIGS. 22E-22F, The roles of canonical ALK5/RSMADs in inhibiting PDK4 expression. Constitutively active ALK5 mutant (ALK5-CA) was adenoviral delivered in HUAECs, and level of phospho-SMAD2, SM22α and PDK4 was showed (FIG. 22E). A reduction of PDK4 level induced by TGFβ2 stimulation, and the protein level of PDK4 was restored by blocking SMAD4 in HUAECs (FIG. 22F).

FIGS. 23A-23C illustrate that Blocking ACSS2, but not ACLY, reverses the effects induced by TGFβ signaling in endothelial cells. FIG. 23A, mRNA level of ACSS2 and PDK genes in HUAECs under different conditions: Scramble siRNA, Scramble siRNA+TGFβ2, or ACSS2 siRNA+TGFβ2. F, CHIP-QPCR testing of Smad-2/3 binding to SBEs on human PDK4 promoter in HUAECs treated with different conditions: Scramble siRNA, Scramble siRNA+TGFβ2, or ACSS2 siRNA+TGFβ2. The data in FIG. 23A were normalized to those of the non-TGFβ treated control or scramble siRNA-treated control and are presented as the mean±SEM from three independent experiments. ****P≤0.0001. FIG. 23B, Representative blots showing the changes of TGFβ-regulated EndMT markers, PDK4 and SMAD2 phosphorylation following overexpression of PDK4 in HUAECs. FIG. 23C, Representative blots showing the changes of ALK1 and ALK5 in HUAECs treated with different conditions: Scramble siRNA, Scramble siRNA+TGFβ2, or ACSS2 siRNA+TGFβ2.

FIGS. 24A-24L illustrate Endothelial Ac-COA is largely derived from acetate in an ACLY-independent manner, and acetate drives EndMT (FIG. 24A) Schematic representation of the stable isotope tracing experimental design. Confluent endothelial colony-forming cells (ECFCs) were pre-treated in tracing medium with unlabeled substrates for 2 h before being switched to labeled tracing medium. (FIG. 24B) Concentration of extracellular 12C-acetate, 13C2-acetate, and total acetate (sum of ¹²C-acetate and ¹³C2-acetate) when ECFCs were exposed to media containing 100 mM of 13C2-acetate. n=3. (C) Acetate uptake over 24 h at varying concentrations of 13C2-acetate. Acetate concentration in medium was normalized to packed cell volume in mL. n=3. (FIG. 24D) Acetate released (i.e., extracellular 12C-acetate) over 24 h at varying concentrations of starting 13C2-acetate. Acetate concentration was normalized to packed cell volume in mL. n=3. (FIG. 24E) Measurement of indicated acetate stable isotope in the cell culture medium after incubation with either U-13C-glucose, U-13C-glutamine, ¹³C2-acetate, or U-¹³C-palmitate for 24 h. Only incubation with U-13C-glucose led to appearance of extracellular 13C2-acetate. n=5-6. (FIG. 24F) Measurement of glucose-derived acetate release (¹³C2-acetate from U-¹³C-glucose) upon ACLY KO with two different sgRNAs over 24 h. n=3. Single pair-wise comparison between two groups (FIGS. 24C-24F) performed by unpaired, two-tailed Student's t test using Prism 9. NS, no significance. **p values<0.01. ***p values<0.001. ****p values<0.0001. (FIG. 24G) Percent isotopolog enrichment of acetyl-CoA when ECFCs are exposed to 5 mM U-13C-glucose, 500 mM U-13C-glutamine, 100 mM 13C2-acetate, and 100 mM U-13C-palmitate for 24 h. The enrichment from 13C2-acetate is corrected for label dilution by intracellular acetate release. n=3. (FIG. 24H) Percent isotopolog enrichment of acetyl-CoA when ECFCs are exposed to varying concentrations of 13C-acetate over 24 h. Enrichment from 13C2-acetate is corrected for media label dilution. n=3. (FIG. 24I) Same as in (FIG. 24G), but for succinyl-CoA. (FIG. 24J) Percent isotopolog enrichment of M+2 acetyl-CoA from 100 mM 13C2-acetate upon ACSS2 KO with two different sgRNAs. Enrichment from 13C2-acetate is corrected for media label dilution. N=3. (FIG. 24K) Percent isotopolog enrichment of M+2 acetyl-CoA from 100 mM 13C2-acetate upon ACLY KO with two different sgRNAs. Enrichment from 13C2-acetate is corrected for media label dilution. n=3. (FIG. 24L) 13C2-acetate released from U-13C-glucose upon TGFβ2 treatment at time points indicated. Acetate concentration in media was normalized to cell number. n=3.

FIGS. 25A-25C illustrate that a blockade of proliferation is induced by blocking PDK4 and sodium acetate treatment. FIG. 25A, EdU positive cells were analyzed in HUAECs transduced with Scramble siRNA or PDK4 siRNA for 7 days. FIG. 25B, EdU staining showing the proliferation of HUAECs treated with different concentration of sodium acetate. FIG. 25C, The pH of the EGM-2 medium containing different concentration of sodium acetate was measured using a pH meter (pHTestr 50S, OAKTON). D-F, Images showing the cell morphology (FIG. 25D). Single pair-wise comparison between two groups performed by unpaired, two-tailed Student's t-test using Prism 9. *P values<0.05. ***P values<0.001. ****P values<0.0001.

FIGS. 26A-26K illustrate ACSS2-generated Ac-COA regulates TGFβ signaling (FIGS. 26A and 26B) Cytosolic and nucleic R-SMADs examined by western blotting (FIG. 26A) and immunostaining (FIG. 26B) in HUACs treated with Scramble siRNA, Scramble siRNA+TGFβ2, or ACSS2 siRNA+TGFβ2 respectively. Scale bars, 25 mm. (FIG. 26C) Representative blots of nucleic R-SMADs in HUAECs treated with Scramble siRNA, ACLY siRNA, or ACSS2 siRNA separately. (FIG. 26D) Representative blots of acetylated R-SMADs in HUAECs transduced with Scramble siRNA or ACSS2 siRNA. Acetylated proteins were captured using agarose beads conjugated with anti-acetylated lysine antibody, and acetylated R-SMADs (Ac-SMAD2/3/4) were detected using anti-SMAD2/3 antibody and anti-SMAD4 antibody.

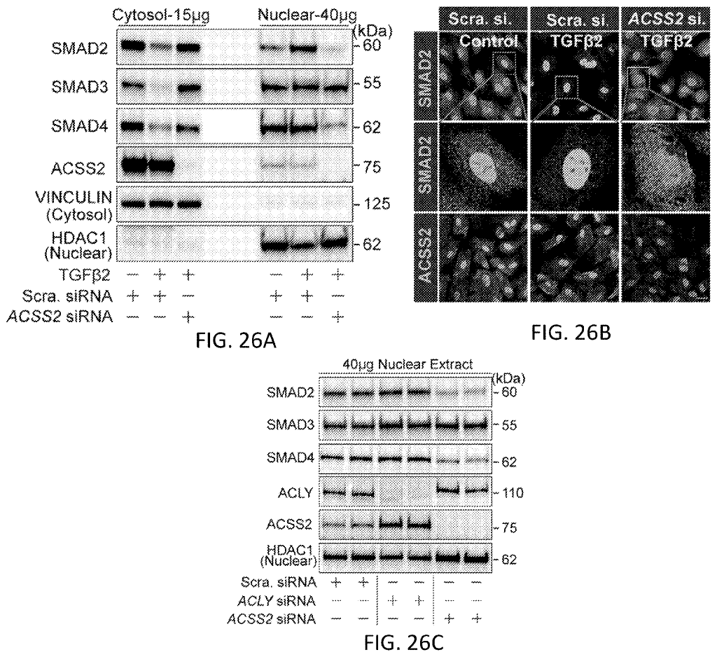

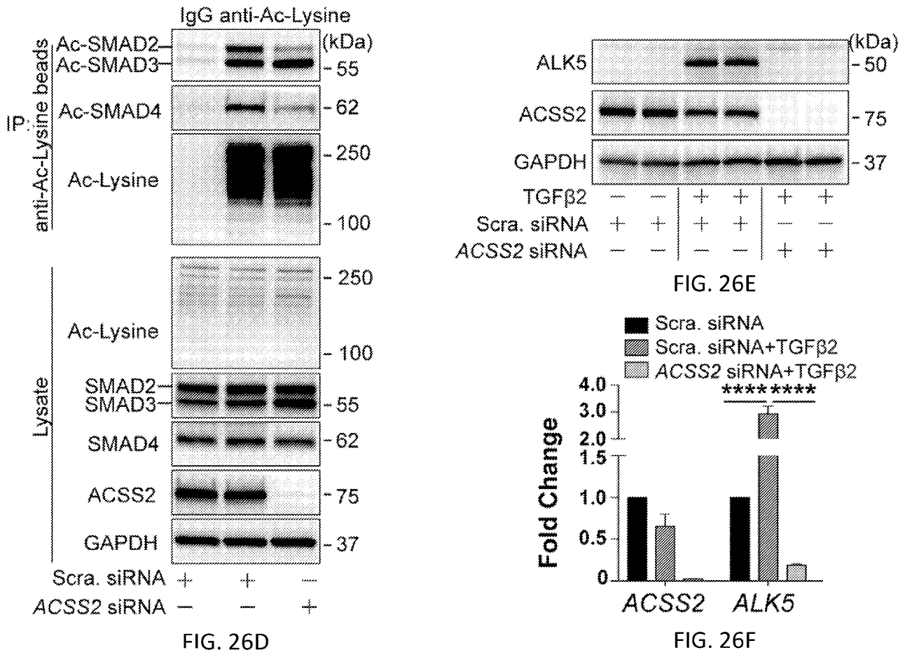

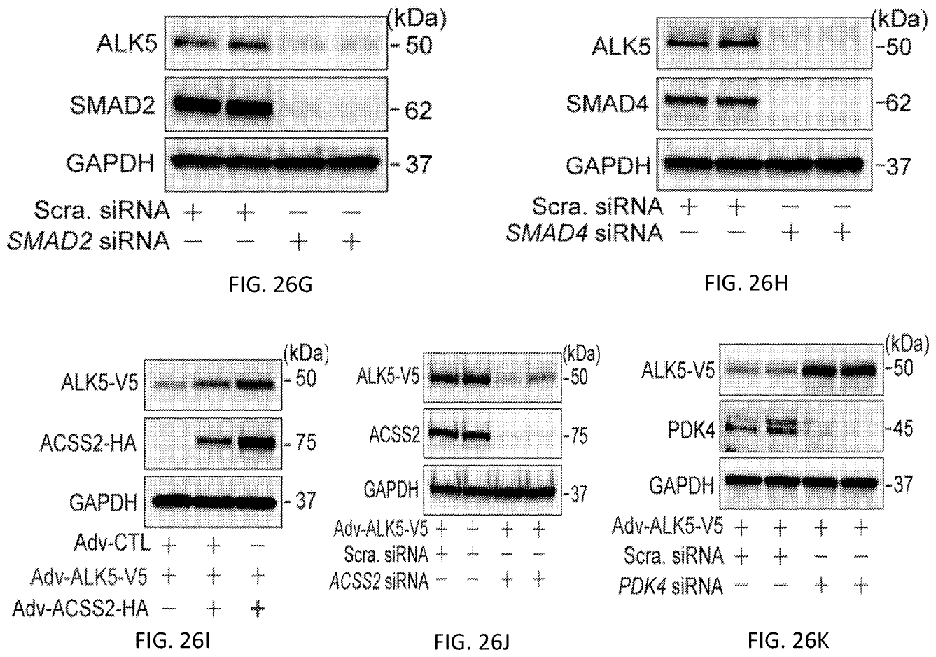

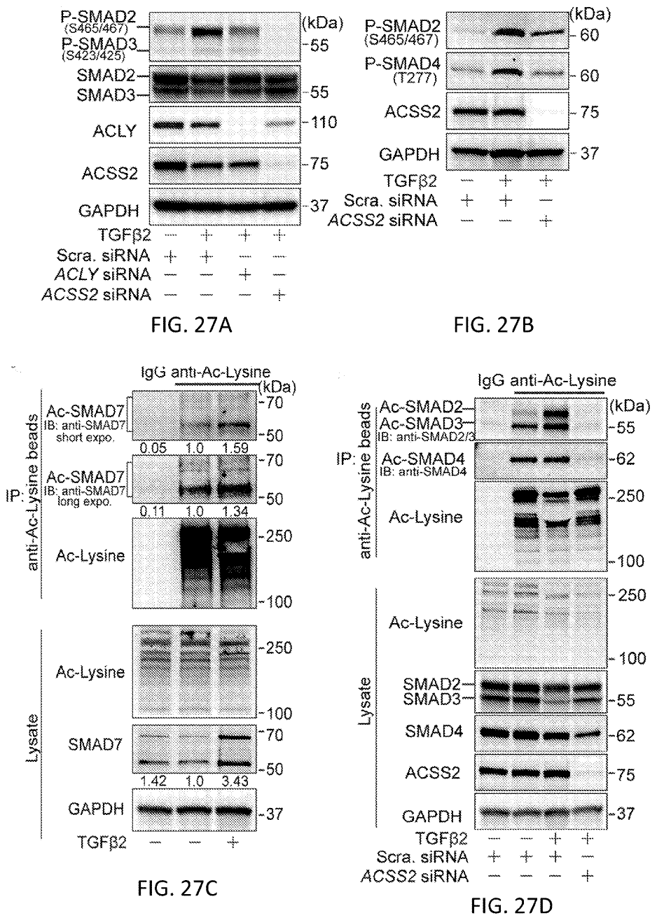

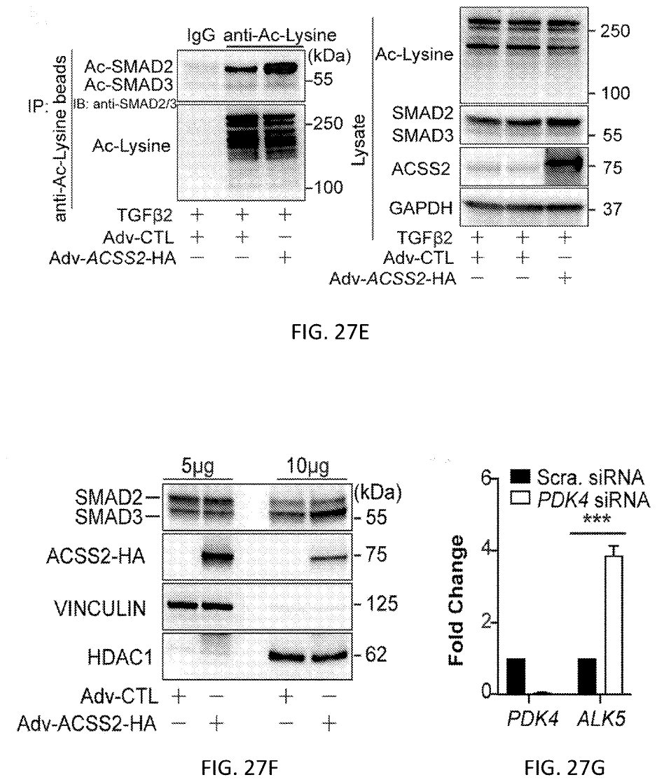

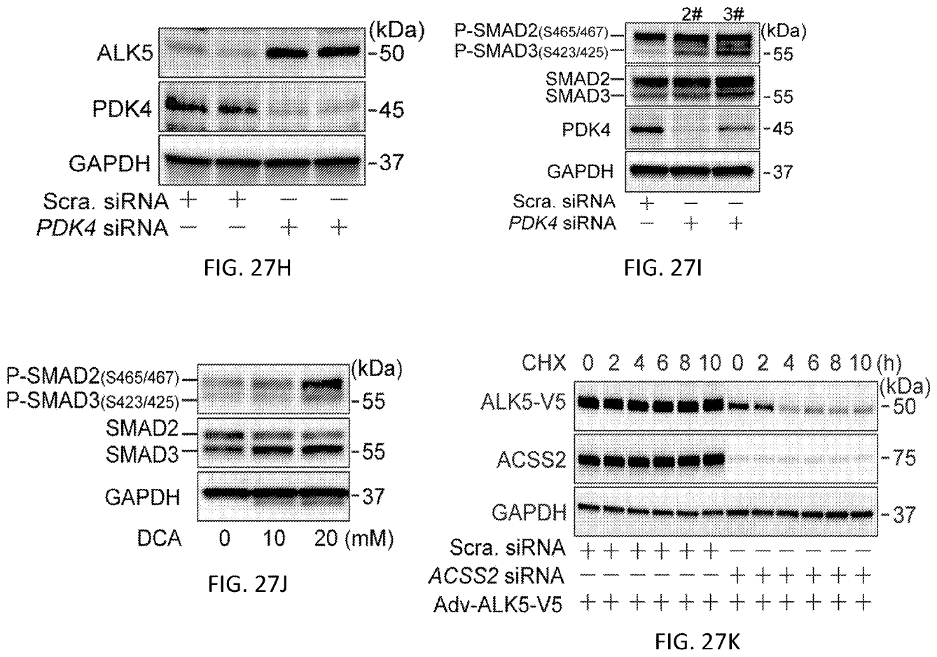

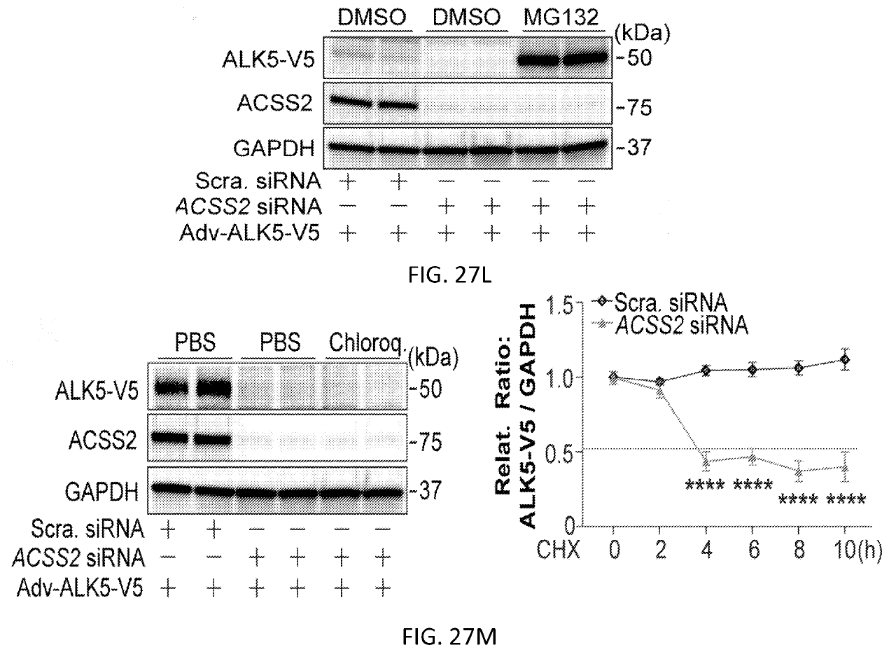

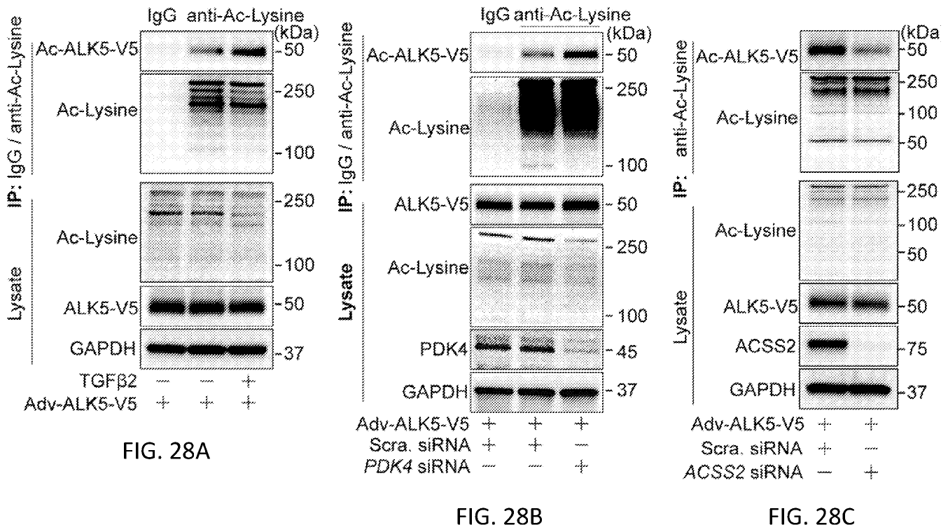

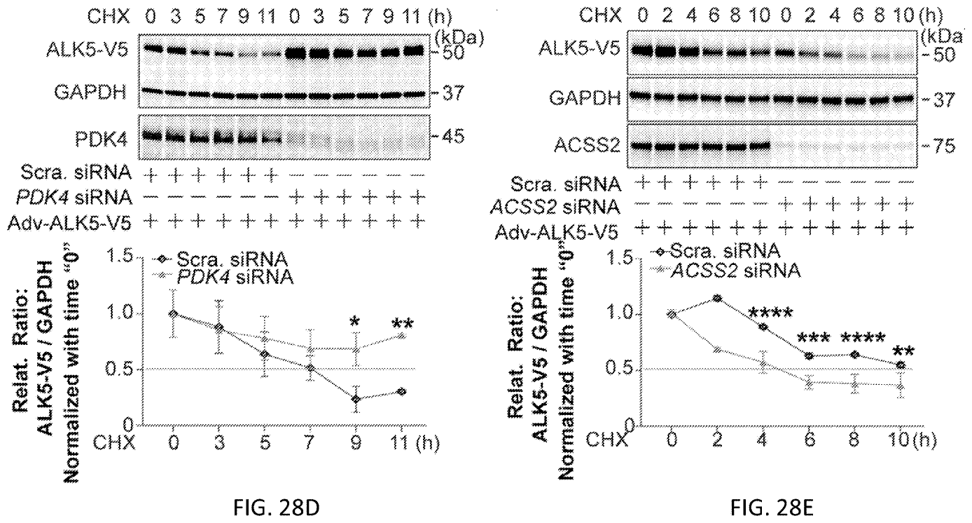

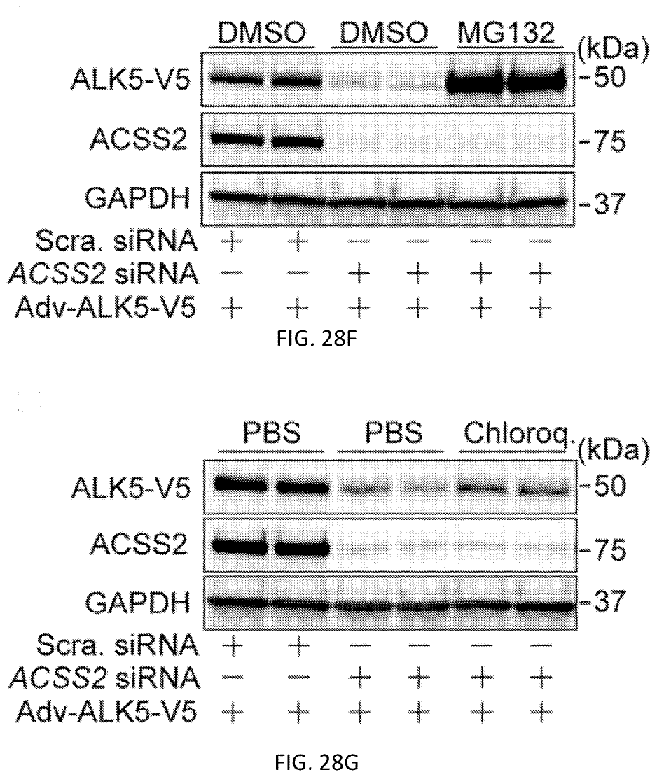

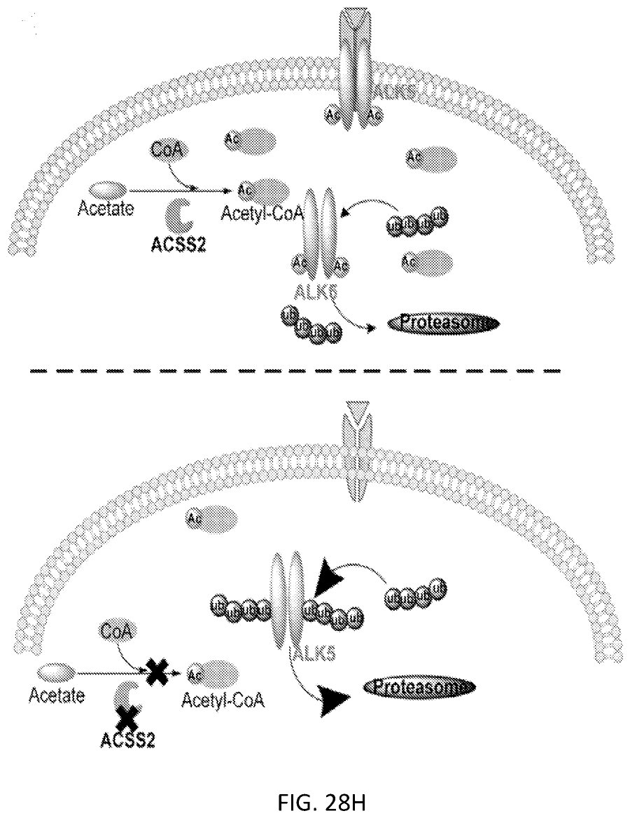

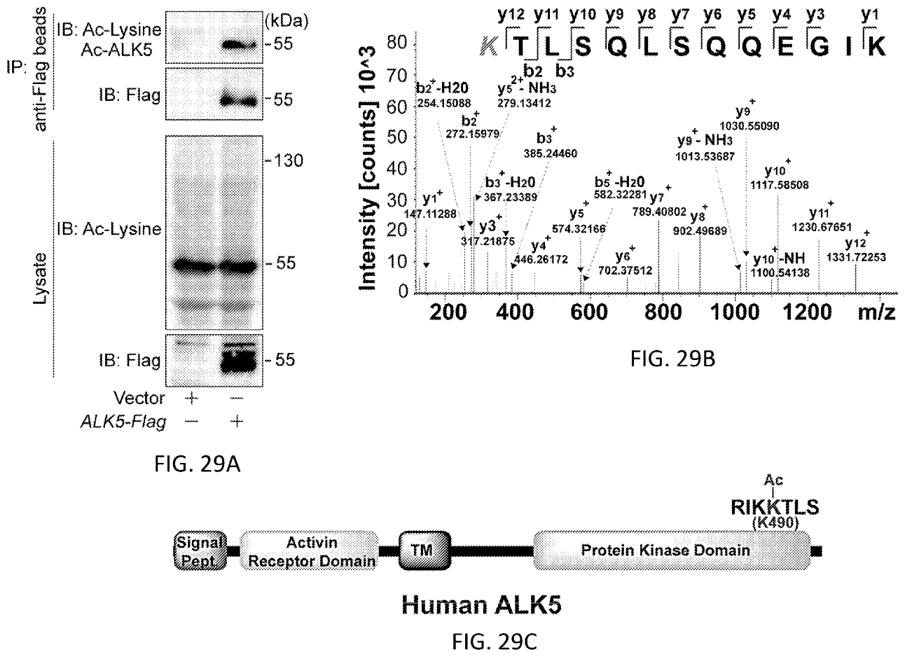

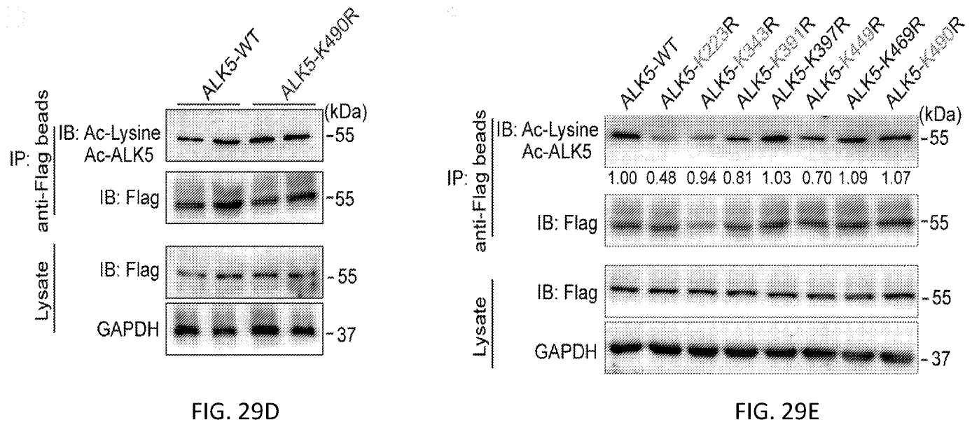

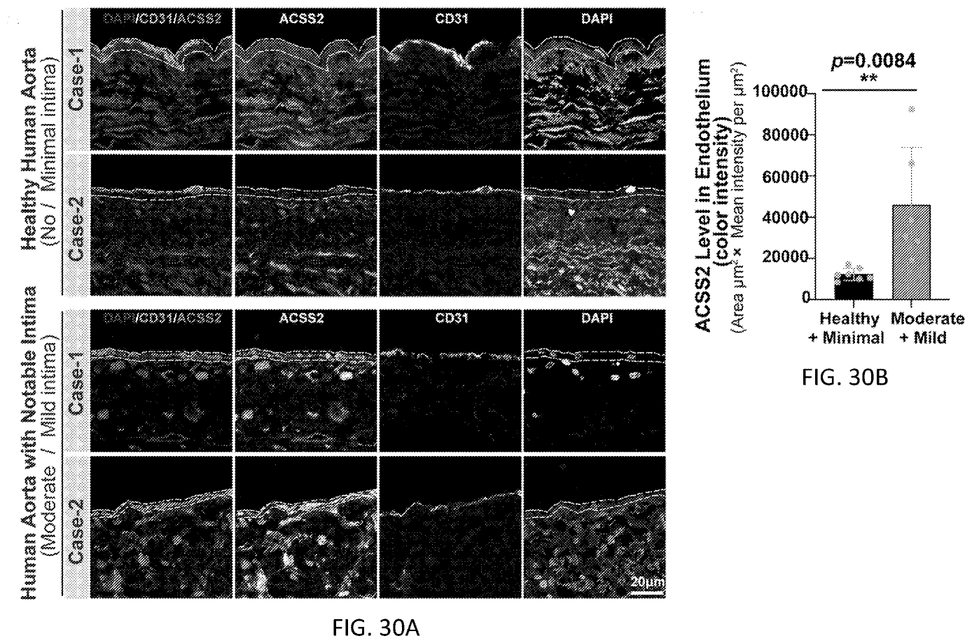

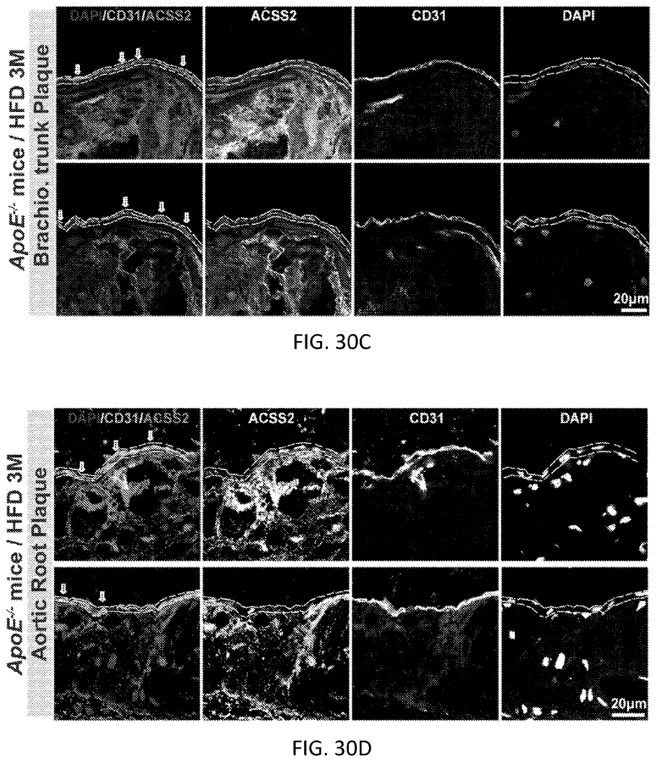

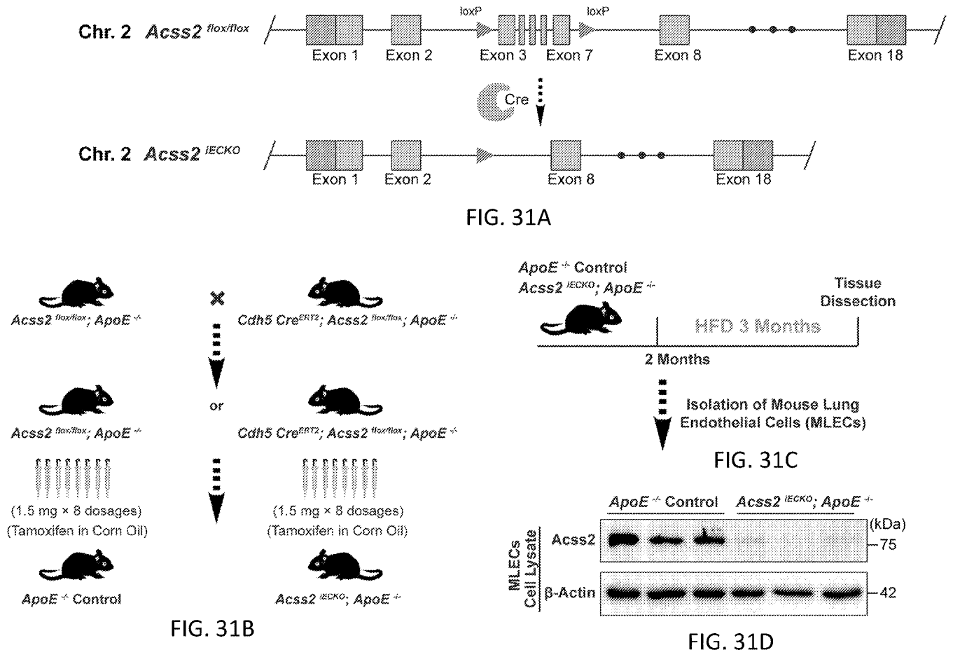

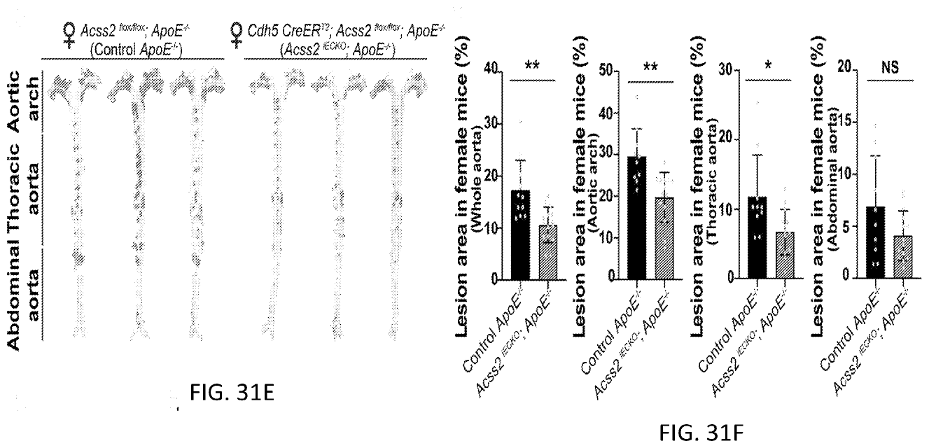

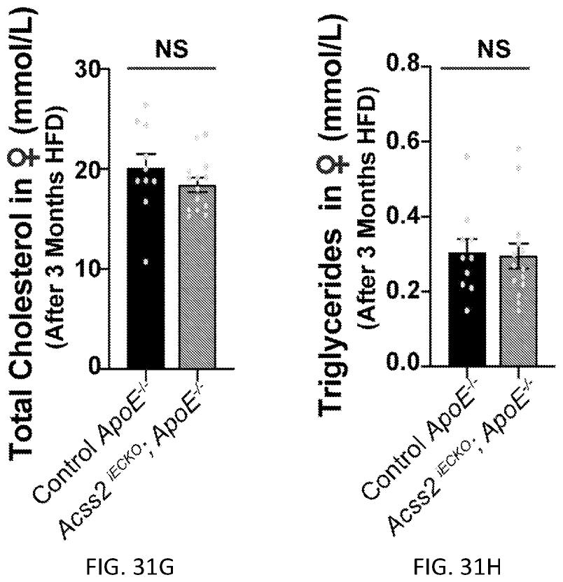

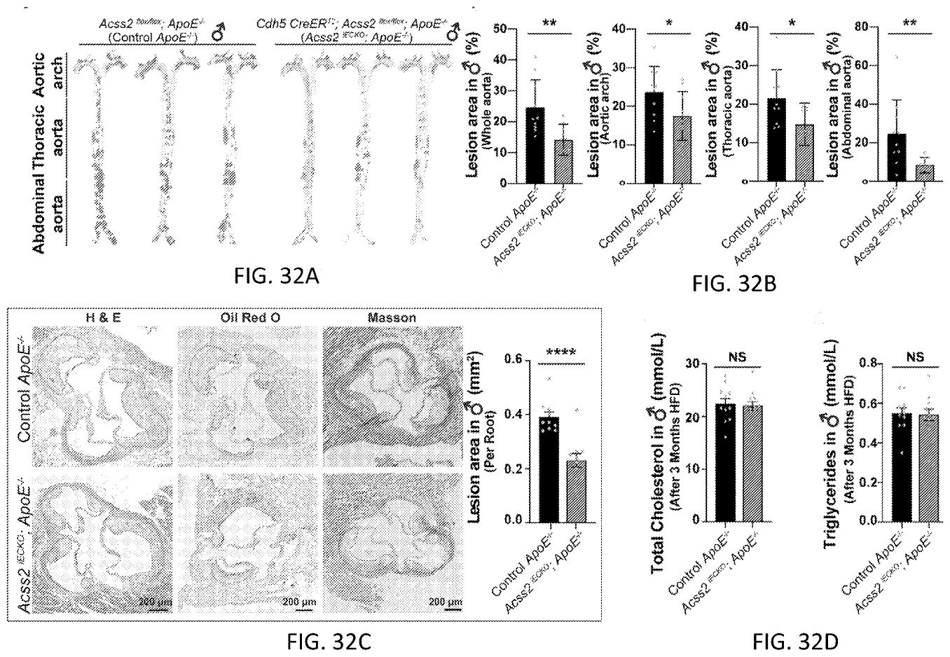

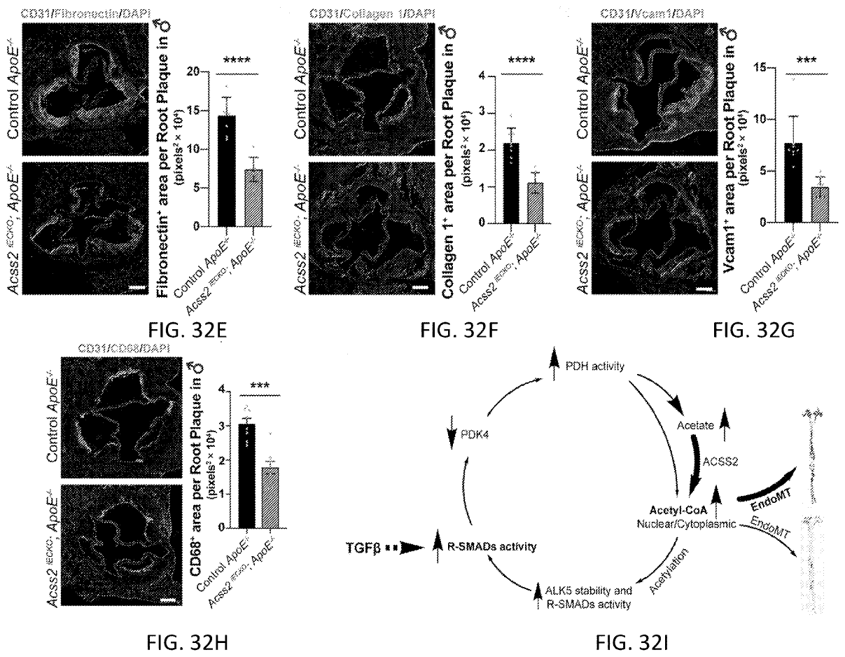

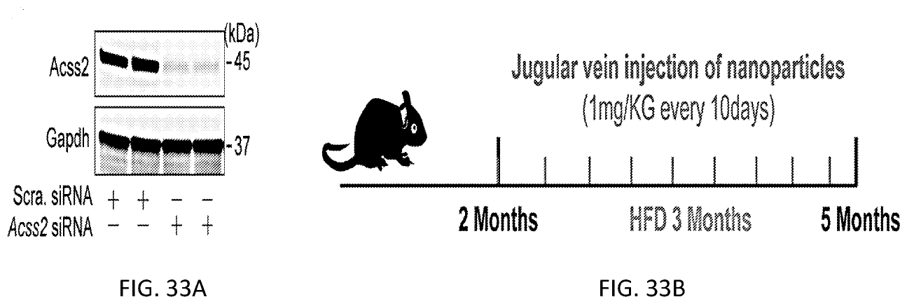

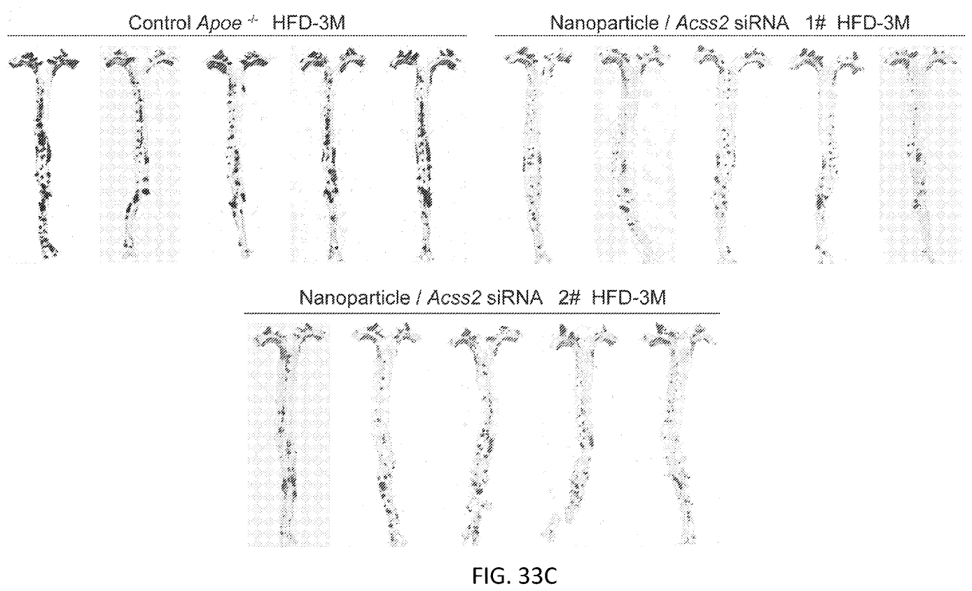

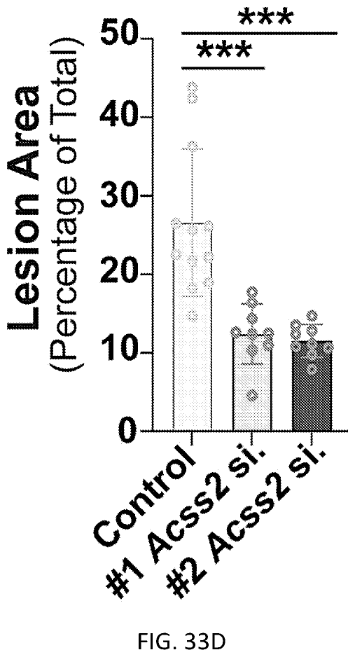

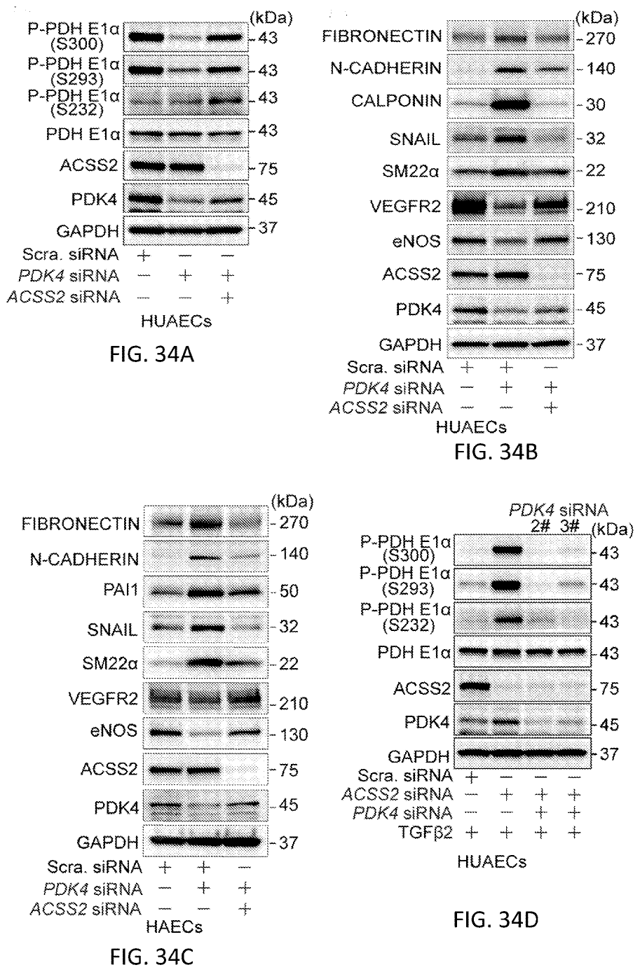

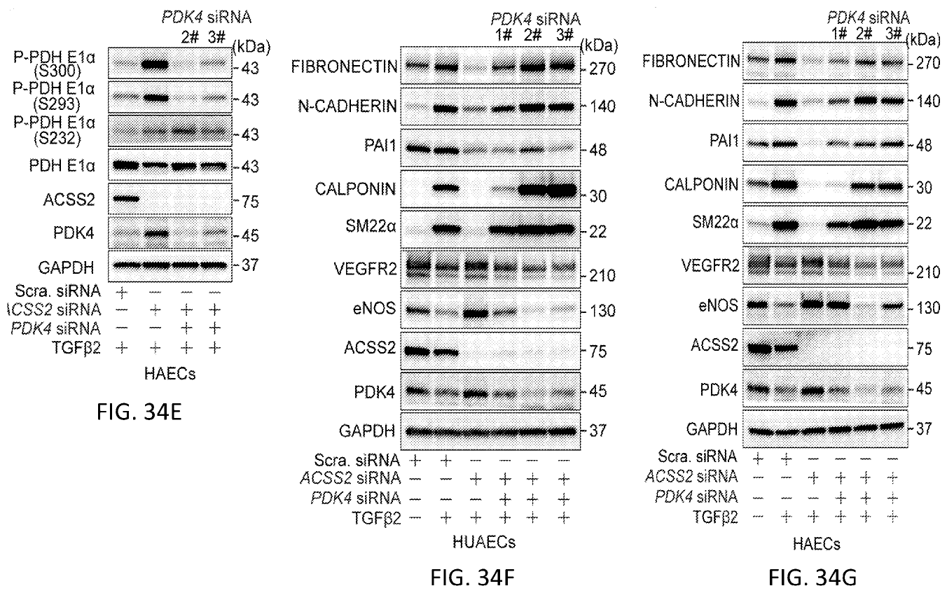

(FIG. 26E) Blots showing the expression of ALK5 in HUAECs transduced with Scramble siRNA or ACSS2 siRNA in the presence of TGFβ2 for 7 days. (FIG. 26F) mRNA level of ALK5 in HUAECs under different conditions: Scramble siRNA, Scramble siRNA+TGFβ2, or ACSS2 siRNA+TGFβ2, respectively. (FIG. 26G) Blots of ALK5 level in HUAECs following with SMAD2 deficiency for 4 days. (FIG. 26H) Blot showing ALK5 level in HUAECs transduced with Scramble siRNA or SMAD4 siRNA for 4 days. (I) Representative blots of V5-tagged ALK5 (ALK5-V5) protein level after gradient overexpression of ACSS2-HA in HUAECs using adenoviral strategy (FIG. 26I). (FIGS. 26J and 26K) Representative blots of ALK5-V5 in HUAECs transfected with Scramble siRNA or ACSS2 siRNA (FIG. 26J)/PDK4 siRNA (FIG. 26K) separately for 4 days. ALK5-V5 was adenoviral overexpressed in HUAECs on the next day after siRNA transfection.

FIGS. 27A-27M illustrate that Acetate-generated Ac-COA mediated by ACSS2 plays positive roles in the maintenance of endothelial TGFβ-ALK5-RSMADs signaling. FIGS. 27A-27B, Representative blots showing phosphorylated R-SMADs in HUAECs (A) and HAECs(FIG. 27B) transduced with Scramble siRNA, ACLY siRNA, or ACSS2 siRNA separately in the presence of TGFβ2 stimulation for 7 days. FIG. 27C, Blots of the total SMAD7 and acetylated SAMD7 in HUAECs treated with TGFβ2 stimulation for 7 days. FIGS. 27D-27E, Blots showing acetylated R-SMADs in HUAECs transduced with Scramble siRNA or ACSS2 siRNA separately in the presence of TGFβ2 stimulation for 7 days (FIG. 27D), or acetylated R-SMADs in HUAECs overexpressing ACSS2-HA in the presence of TGFβ2 stimulation for 3 days (FIG. 27E). F, Representative blots of cytosolic and nucleic RSMADs in HUAECs overexpressing ACSS2HA for 3 days. FIGS. 27G-H, mRNA (FIG. 27G) and protein (FIG. 27H) level of ALK5 in HUAECs transduced with Scramble siRNA or PDK4 siRNA for 7 days. FIGS. 27I-27J, Blots showing phosphorylated R-SMADs in HUAECs with PDK4 knockdown (FIG. 27I) or pharmacological inhibition of PDKs with DCA (FIG. 27J) for 7 days. K, Half-life of ALK5-V5 in HUVECs transduced with Scramble siRNA or ACSS2 siRNA separately. ALK5-V5 was adenovirally overexpressed in HUVECs followed by cycloheximide (CHX, 10 μg/ml) treatment at the indicated time points. ALK5-V5 protein level was determined by anti-V5 antibody. FIGS. 27L-27M, Representative blots showing the regulation of ALK5 degradation in HUVECs. HUVECs were firstly transduced with Scramble siRNA or ACSS2 siRNA, then ALK5-V5 was adenovirally overexpressed in HUAECs followed the treatment with a proteasome inhibitor MG132 (FIG. 27L) or lysosome inhibitor chloroquine (FIG. 27M).

FIGS. 28A-28H illustrate that ACSS2-generated Ac-COA promotes protein stability of ALK5 (FIG. 28A) Representative blot of acetylated ALK5-V5 in HUAECs treated or non-treated with TGFβ2 (10 ng/mL in complete EGM-2 medium) for 7 days. Acetylated proteins were captured using agarose beads conjugated with anti-acetylated lysine antibody, and acetylated ALK5-V5 (Ac-ALK5-V5) was detected using anti-V5 tag antibody. (FIGS. 28B and 28C) Blots of Ac-ALK5-V5 in HUAECs transduced with Scramble siRNA or PDK4 siRNA (FIG. 28B)/ACSS2 siRNA (FIG. 28C) separately for 7 days. Acetylated proteins were captured and detected as described above. (FIGS. 28D and 28E) Half-life of ALK5-V5 in HUAECs transduced with Scramble siRNA or PDK4 SiRNA (FIG. 28D)/ACSS2 siRNA (FIG. 28E). ALK5-V5 was adenovirally overexpressed in HUAECs cycloheximide (CHX, 10 mg/mL) treatment at indicated time points. ALK5-V5 protein level was determined by anti-V5 antibody. (FIGS. 28F and 28G) Representative blots showing the regulation of ALK5 degradation in HUAECs. HUAECS were first transduced with Scramble siRNA or ACSS2 siRNA, then ALK5-V5 was adenovirally overexpressed in HUAECs followed by treatment with a proteasome inhibitor MG132 (FIG. 28F) or a lysosome inhibitor chloroquine (FIG. 28G). (FIG. 28H) Diagram for Ac-CoA-regulated ALK5 acetylation results in increased protein half-life due to decreased proteasomal degradation.

FIGS. 29A-29E illustrate the characterization of acetylated sites for Human ALK5. FIG. 29A, Representative blots of exogenous acetylated ALK5 in 293T cells transfected with Vector or ALK5-Flag plasmids, ALK5 (Flag) was immunoprecipitated followed by immunoblotting to detect Acetylated-Lysine (Ac-Lysine) and ALK5 (Flag). FIG. 29B, LC-MS/MS analysis of the digested ALK5-Flag proteins immunoprecipitated by anti-Flag beads, and Lysine 490 is the acetylated site of ALK5 by analyzing the peptides of mass spectrum. FIG. 29C, Lysine residue locates at the protein kinase domain of ALK5. FIG. 29D, 293T cells were transfected with ALK5-WT or K490R mutant bearing single lysine (K) to arginine (R) substitutions at Lysine 490 site, and ALK5-WT or K490R mutant were immunoprecipitated with anti-Flag beads. Acetylated-ALK5 protein level was determined by anti-Ac-Lysine antibody. FIG. 29E, Single K-R mutation strategy in characterizing acetylation sites for Human ALK5. There are 25 Lysine residues in Human ALK5, and 25 mutants bearing single K-R mutation were constructed. After IP-anti-FLAG/IB-anti-Ac-Lysine screening analysis, representative blots showing the major lysine residues (K223, K343, K391, K449, and K490 (by LC-MS/MS)) for acetylation.

FIGS. 30A-30D illustrate immunostaining of ACSS2 in human aorta and mice brachiocephalic trunk and aortic root (FIG. 30A) ACSS2 expression was studied in human aortas (n=13) from normal organ donors with minimal or moderate atherosclerosis extent. In all cases, ACSS2 expression was examined in relatively normal aortic segments (no/minimal disease) and in segments demonstrating mild/moderate atherosclerosis as judged by the extent of neointima development. ACSS2 expression (red signal), endothelial cells identified with anti-CD31 (green staining), nuclei identified with DAPI (blue). (B-D) (FIG. 30B) Quantification of ACSS2 expression in endothelial cells areas (as indicated with dashed line) from normal/minimal disease and mild/moderate disease specimens. Statistical analysis was performed by unpaired, two-tailed Student's t test using Prism 9. **p values<0.01. Representative frozen sections of the brachiocephalic trunk (FIG. 30C) and aortic root (FIG. 30D) dissected from control ApoE-/______mice after 3 months of high-fat diet were immunostained with anti-CD31 (green), DAPI (blue), and anti-Acss2 (red) antibodies. Merged channels and a single channel of Acss22/CD31/DAPI were displayed in gray color. And the area of endothelium was indicated with dashed lines.

FIGS. 31A-31H illustrate the generation of Cdh5-CreER mice and Acss2 T2 fl/fl −/−; Acss2; Apoe fl/fl−/− mice. FIG. 31A, diagram showing the genomic information of Acss2 gene in Acss2; Apoe flox/flox mice. FIG. 31B, diagram showing the generation of Acss2 fl/fl−/−; Apoe mice and Cdh5-CreER T2 fl/fl−/−; Acss2; Apoe mice. FIGS. 31C-31D, Deletion efficiency of Acss2 gene in the lung endothelial cells of Cdh5-CreER mice. FIG. 31E, Plaques in aortas of control ApoE T2 fl/fl−/−; Acss2; Apoe−/−mice (♀) were stained with OilRed-O. F, Oil-Red-O analysis of whole aortas, aortic arch, thoracic aorta, and abdominal aorta from control ApoE female mice (♀) and Acss2 iECKO; ApoE−/−female−/− mice (♀) (n=14). FIGS. 31G-31H, total cholesterol level (FIG. 31G) and triglycerides (FIG. 31H) in plasma collected from control ApoE female mice (♀) (n=10) and Acss2 iECKO; ApoE−/− female female mice (♀) and Acss2−/− female mice (♀) after 3 months of high fat diet. NS, no significance. The plaque lesion area and total surface area of aortas were quantified using ImageJ software. A single pair-wise comparison between two groups performed by unpaired, two-tailed Student's t-test using Prism 9. NS, no significance, *P values<0.05, **P values<0.01.

FIGS. 32A-32J illustrate Reduced development of atherosclerosis after endothelial-specific deletion of Acss2 in ApoE−/− mice (FIG. 32A) Plaques in aortas of control ApoE−/− mice (male) and Acss2 iECKO; ApoE−/− mice (male) were stained with oil red O. (FIG. 32B) Oil red O analysis of whole aortas, aortic arch, thoracic aorta, and abdominal aorta from control (n=10) and Acss2 iECKO; ApoE−/− mice (male) (n=10). The plaque lesion area and total surface area of aortas were quantified using ImageJ software. A single pair-wise comparison between two groups performed by unpaired, two-tailed Student's t test using Prism 9. *p values<0.05, **p values<0.01. (FIG. 32C) Plaques in aortic roots of control ApoE−/− mice and Acss2 iECKO; ApoE−/− mice were analyzed with H&E staining, oil red O staining, and Masson staining. The area of the plaque per root were quantified. A single pair-wise comparison between two groups performed by unpaired, two-tailed Student's t test using Prism 9. ****p values<0.0001. (FIGS. 32D and 32E) total cholesterol level (FIG. 32D) and triglycerides (FIG. 32E) in plasma collected from control ApoE−/− mice and Acss2 iECKO; ApoE−/− mice after 3 months of high-fat diet. NS, no significance. (FIGS. 32F-32I) immunostaining of Fibronectin 1 (FIG. 32F), Collagen 1 (FIG. 32G), Vcaml (FIG. 32H), andCD68 (FIG. 32I) on aortic root sections from both control ApoE−/− mice and Acss2 iECKO; ApoE−/− mice. A single pair-wise comparison between two groups performed by unpaired, two-tailed Student's t test using Prism 9. ***p values<0.001, ****p values<0.0001. (FIG. 32J) Diagram of metabolic reprogramming regulated by TGFβ signaling in EndMT. TGFβ signaling promotes glucose uptake and glycolysis in endothelial cells and increases the activity of pyruvate dehydrogenase complex (PDC) by suppressing PDK4 expression. TGFβ signaling induces glucose conversion to acetate which is then followed by the conversion of acetate to cytosolic acetyl-CoA mediated by ACSS2, ultimately leading to increased activity of R-SMADs and ALK5 subsequently that, in turn, further promotes TGFβ signaling there by establishing a positive-feedback loop. Blocking the acetate conversion to Ac-COA by ACSS2 knockdown decreased the acetylation of R-SMADs and ALK5 and disrupts TGFβ signaling thereby interrupting in this positive-feedback loop.

FIGS. 33A-33D illustrate ACSS2 knockdown in ApoE mice. FIG. 33A. A knockdown efficiency of ACSS2 siRNA in mice artery endothelial cells (MAEC). MAECs were incubated with 20 nM unmodified ACSS2 siRNAs in EGM-2/Opti-MEM medium for 10 hrs. Three days after siRNA delivery, Acss2 level in total cell lysates was analyzed by immunoblotting with anti-ACSS2 antibody. FIG. 33B, Flow diagram showing timing of atherosclerosis induction and 7C1 LNP delivery in ApoE−/− −/− mice were stained with Oil-Red-O. Images showing the Oil-Red-O staining of aortas dissected from 12 control ApoE mice. FIGS. 33C-33D, Plaques in aortas of ApoE−/− mice and 9 unmodified (siRNA 1) and 9 modified (siRNA 2) ACSS2 siRNA-treated ApoE−/− mice (FIG. 33C). OilRed-O analysis of whole aortas from control and ACSS2 siRNA-treated ApoE−/− mice. The plaque lesion area and total surface area of aortas were quantified using ImageJ software. A single pair-wise comparison between two groups performed by unpaired, two-tailed Student's t-test using Prism 9. ***P values<0.001 (FIG. 33D).

FIGS. 34A-34G illustrate that ACSS2 and inhibition of PDK4 drive a positive feedback loop. FIG. 34A, Representative blots showing the phosphorylation of PDHE1αdecreased by PDK4 knockdown can be restored by ACSS2 deficiency in HUAECs. FIGS. 34B-34C, Representative blots showing ACSS2 knockdown reduced EndMT markers and restored EC-specific gene expression induced by PDK4 siRNA in HUAECs (FIG. 34B) and HAECs (FIG. 34C). FIGS. 34D-34E, In the context of pro-EndMT TGFβ signaling, the elevated phosphorylation of PDHE1α by ACSS2 knockdown was reversed by knocking down PDK4 in HUAECs (FIG. 34D) and HAECs (FIG. 34E). F-G, In the presence of TGFβ signaling, the reduced EndMT markers and restored EC-specific gene expression by ACSS2 knockdown can be reversed by blocking PDK4 in HUAECs (F) and HAECs (FIG. 34G).

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

“Allogeneic” refers to any material derived from a different animal of the same species.

“Xenogeneic” refers to any material derived from an animal of a different species.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” “an autoimmune disease-inhibiting effective amount,” or “therapeutic amount” is indicated, the precise amount of the compositions of the present invention to be administered can be determined by a physician or researcher with consideration of individual differences in age, weight, tumor size, extent of infection or metastasis, and condition of the patient (subject).

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject. The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, at least in part, on the discovery that inhibition of Acetyl COA Synthetase Short chain family member 2 can inhibit the process of endothelial-to-mesenchymal transition (EndMT) in endothelial cells. The process of EndMT is a key pathological event in chronic vascular inflammatory diseases, including atherosclerotic cardiovascular disease, pulmonary hypertension, and vascular fibrotic states. In endothelial cells, the molecular mechanism of EndMT progression is driven by TGFβ signaling, which results in the increased production of acetyl-CoA, a key molecule in metabolic processes. Production of acetyl-CoA via TGFβ depends on the activity of the enzyme Acetyl COA Synthetase Short chain family member 2 (ACSS2). Experimental evidence obtained herein confirms that blocking or inhibiting the function of ACSS2 not only blocks production of acetyl COA during TGFβ signaling, but can block the induction of EndMT. As such, in some aspects, the current invention includes methods and compositions useful for inhibiting EndMT comprising an effective amount of a ACSS2 inhibitor. In certain aspects, the invention also includes methods of treating a vascular inflammatory disease in a subject in need thereof comprising administering an effective amount of an inhibitor of ACSS2. In certain embodiments, the ACSS2 inhibitor is an inhibitor RNA. In certain embodiments, the ACSS2 inhibitor is a small molecule. In certain embodiments, the ACSS2 inhibitor is a DNA editing system which targets the ACSS2 gene. In certain embodiments, the DNA editing system is a CRISPR/Cas9 system.

### Endothelial to Mesenchymal Transition

Endothelial to mesenchymal transition (EndMT) is an intricate cellular differentiation process that involves phenotypic change, proliferation and migration of endothelial cells into the neointima. Thus transformed, endothelial cells become highly inflammatory and promote constant inflammation in the vascular wall that, in turn, drives further EndMT. This establishes a “vicious circle” with EndMT begetting inflammation begetting EndMT. The presence of chronic vascular inflammation is the key factor leading to atherosclerosis and other vascular diseases mentioned above. While EndMT is a vital process during embryonic development, it can also be induced as a result of persistent damage and tissue inflammation of vascular tissues. Additionally, persistent EndMT can lead to severe and even complete organ fibrosis or development of a pre-malignant stroma when associated with angiogenesis.

EndMT not only drives the accumulation of “mesenchymal type” (smooth muscle, fibroblasts) cells into atherosclerotic plaque structures, but induces further inflammatory activation of luminal endothelial cells, extracellular matrix remodeling, and increased permeability. These events promote further entry and retention of both leukocytes and lipoproteins, which promote further inflammation and further EndMT, thereby creating a self-sustaining feed-forward loop. Once set in motion, this process continues even if initiating factors are no longer present. Described herein are methods to inhibit EndMT in order to arrest or induce regression of established cardiovascular disease as therapeutic strategy.

EndMT occurs in various inflammatory conditions, including transplant arteriopathy, a relentless disease that is the primary reason for long-term failure of various organ drugs, such as for the heart or kidneys. There are no known therapies for this condition. EndMT is also important driver of in pulmonary hypertension and various conditions associated with chronic inflammation-induced fibrosis such as scleroderma, Systemic Lupus Erythematosus (SLE), hepatic fibrosis and other fibrosis states and the like to name a few. Accordingly, without being bound by theory, the same treatment that is effective in reducing EndMT is expected to be effective in treatment of the foregoing diseases.

Acyl-coenzyme A synthetase short-chain family member 2 (ACSS2) is a cytosolic enzyme that catalyzes the activation of acetate for use in lipid synthesis and production of energy. ACSS2 is a monomer that produces acetyl-CoA from acetate in a reaction that requires ATP. In the studies of the current disclosure, ACSS2 is shown to be the major player in the acetyl-CoA production that occurs as a consequence of TGFβ signaling in EndMT. Here TGFβ inhibits the expression of PDK4, which in turn releases regulation of the PDH complex, which leads to the generation of acetate, which is acted upon by ACSS2 to produce acetyl-CoA. Accordingly, in some embodiments, the present invention includes the inhibition of ACSS2 activity in order to block or prevent the process of EndMT.

### Methods of Treatment

In some aspects, the present invention provides a method of treating a vascular inflammatory disease and/or disorders or symptoms thereof which comprise administering a therapeutically effective amount of a pharmaceutical composition comprising an agent that modulates the activity or level of ACSS2 in a cell, to a subject (e.g., a mammal such as a human).

In particular embodiments, the agent that modulates the activity or level of ACSS2 inhibits the activity or level of ACSS2 in a cell. In some embodiments, the cell is an endothelial cell. In certain embodiments, the agent that increases the activity or level of ACSS2 in a cell is a ACSS2-targeting inhibitory RNA. In some other embodiments, the agent is a siRNA. In some embodiments, the ACSS2 inhibitor is a small molecule. In some embodiments, the agent that decreases the activity or level of ACSS2 in a cell is an inhibitory polynucleotide that reduces expression of ACSS2 protein.

In other aspects of the invention, the agent that decreases the activity or level of ACSS2 protein is a nucleic acid capable of downregulating the gene expression of ACSS2.

In some instance, downregulation of the ACSS2 gene expression is desired. This downregulation may result from a full or partial knock down of the gene of interest. Briefly, a gene knock down refers to a genetic technique in which one of an organism's genes is silenced, made inoperative or partially inoperative. Gene expression may be downregulated, knocked-down, decreased, and/or inhibited by various well-established molecular techniques known in the art such as, but not limited to, RNA interference (RNAi), small inhibitor RNA (siRNA), small hairpin RNA (shRNA) and Clustered Regularly Interspaced Short Palindromic Repeats (CRISPRs)).

In some embodiments, the nucleic acid is selected from the group consisting of an antisense RNA, siRNA, shRNA, and a CRISPR system. In other embodiments, the nucleic acid is encapsulated in a nanoparticle formulated for selective delivery to an endothelial cell, in a pharmaceutically acceptable excipient. A number of nanoparticle formulations are suitable for the delivery of the nucleic acids of the invention are known in the art. Non-exclusive examples of lipids used for nanoparticle formation include DSPC, 1,2-distearoyl-sn-glycero-3-phosphorylcholine; DLin-MC3-DMA, (6Z,9Z,28Z,31Z)-Heptatriaconta-6,9,28,31-tetraen-19-yl4-(dimethylamino)butanoate; PEG-DMG, (R)-2,3-bis(octadecyloxy) propyl-1-(methoxy polyethylene glycol 2000) carbamate; and PEG-DSG, (R)-2,3-bis(stearyloxy) propyl-1-(methoxy poly(ethylene glycol) 2000 carbamate. In particular embodiments, the nanoparticle is a 7C1 nanoparticle, which is useful for delivering RNAs to endothelial cells and has been described in Dahlman, et al. (2014) Nature Nanotechnology 8:648-655.

TABLE 1
ACSS2-targeting siRNAs used in the invention.

| SEQ | | |
|---|---|---|
| ID NO: | Name: | Sequence: |
| 1. | NO-1 Sense | CAGCAAUGUUCUCCGUAAAtt |
| 2. | NO-1 Antisense | tgGUCGUUACAAGAGGCAUUU |
| 3. | NO-2 Sense | CUGAUAUGCUCCUCGACUAtt |
| 4. | NO-2 Antisense | tgGACUAUACGAGGAGCUGAU |
| 5. | NO-3 Sense | GAUCAUGCGCACAGUCUAUtt |
| 6. | NO-3 Antisense | tgCUAGUACGCGUGUCAGAUA |
| 7. | NO-4 Sense | CUGCACUAGGCUUUAGAAAtt |
| 8. | NO-4 Antisense | tgGACGUGAUCCGAAAUCUUU |

The methods disclosed herein include administering to the subject (including a subject identified as in need of such treatment) an effective amount of an agent described herein, or a composition described herein to produce such effect. Identifying a subject in need of such treatment can be made by a health care professional and may be subjective (e.g. opinion) or objective (e.g. measurable by a test or diagnostic method, such as using the methods described herein).

The therapeutic methods of the invention, which may also include prophylactic treatment, in general comprise administering a therapeutically effective amount of one or more of the agents herein (such as an agent that modulates the activity or level of ACSS2,) to a subject (e.g., animal, human) in need thereof, including a mammal, particularly a human. Such treatment is suitable for subjects, particularly humans, suffering from, having, susceptible to, or at risk for a chronic vascular inflammatory disease, disorder, or symptom thereof. In certain embodiments the vascular inflammatory disease is selected from the group consisting of atherosclerosis, myocardial infarction, pulmonary hypertension, and stroke. It is also contemplated that the methods of the invention could be used to treat a vascular inflammatory disease associated that results from a vascular graft failure, including but not limited to neointimal stenosis and the like.

In certain embodiments, the methods of the invention can be used to treat a vascular inflammatory disease associated with a fibrotic state of the vessels.

### Pharmaceutical Compositions

The present invention features compositions useful for inhibiting ACSS2 in a subject. The compositions include an agent that modulates the activity or level of ACSS2 in a cell.

In particular embodiments, the agent that modulates the activity or level of ACSS2 inhibits the activity or level of ACSS2 in a cell, in particular, an endothelial cell. In certain embodiments, the agent that decreases the activity or level of ACSS2 in a cell is an inhibitory RNA that reduces the expression of ACSS2 protein. In some other embodiments, the agent is a polynucleotide encoding a ACSS2 inhibiting RNA that reduces the expression of ACSS2. In some embodiments, the agent that decreases the activity or level of ACSS2 in a cell is a small molecule.

In certain aspects, the invention includes compositions comprising the agents that modulate the activity or level of ACSS2 in a cell. In certain embodiment, the compositions of the invention may be administered systemically, for example, formulated in a pharmaceutically-acceptable buffer such as physiological saline. Routes of administration include, for example, subcutaneous, intravenous, intraperitoneally, intramuscular, or intradermal injections that provide continuous, sustained levels of the agent in the patient. In certain embodiments, the compositions of the invention are formulated in nanoparticles, including lipid nanoparticles such that the ACSS2 modulating agents can be efficiently delivered to cells. In certain embodiments, the lipid nanoparticles possess tissue specificity. In certain embodiments the lipid nanoparticles preferentially target endothelial cells.

The amount of the therapeutic agent to be administered varies depending upon the manner of administration, the age and body weight of the patient, and with the clinical symptoms of chronic vascular inflammation. Generally, amounts will be in the range of those used for other agents used in the treatment of atherosclerosis, although in certain instances lower amounts will be needed because of the increased specificity of the agent. A composition is administered at a dosage that decreases effects or symptoms of atherosclerosis as determined by a method known to one skilled in the art.

The therapeutic agent may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target atherosclerosis using carriers or chemical derivatives to deliver the therapeutic agent to a particular cell type (e.g., endothelial cells or smooth muscle cells). For some applications, controlled release formulations obviate the need for frequent dosing during the day to sustain the plasma level at a therapeutic level. Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The pharmaceutical composition of this invention could be coated or comprised in a drug-eluting stent (DES) ((Nikam et al., 2014 Med Devices 7:165-78)) that releases at a given site (such as an artery) and pace (i.e. slow release) the composition of this invention. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates atherosclerosis, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

In some embodiments, the composition of this invention is delivered locally from, but not limited to, the strut of a stent, a stent graft, a stent cover or a stent sheath. In some embodiments, the composition of this invention comprises a rapamycin or a derivative thereof (e.g. as described in U.S. Pat. No. 6,273,913 B1, incorporated herein by reference). In some embodiments, the composition comprising the active therapeutic is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Polynucleotide Therapy

In some embodiments, the invention includes a method for treating, slowing the progression of, or reversing a chronic vascular inflammatory disease, where a therapeutic polynucleotide inhibitor of ACSS2 is administered to the subject. In certain embodiments, the polynucleotide is an inhibitory polynucleotide that reduces expression of ACSS2 protein. Inhibitory polynucleotides include, but are not limited to siRNAs that target a polynucleotide encoding ACSS2 protein.

As described elsewhere herein, ACSS2 regulates the production of acetyl-CoA in response to TGFβ signaling in endothelial cells and contributes to the process of EndMT, thereby suppressing ACSS2 activity can resist the progression of EndMT and treat the chronic vascular inflammatory disease.

Such therapeutic polynucleotides can be delivered to cells of a subject having a chronic vascular inflammatory disease. The nucleic acid molecules are delivered to the cells of a subject in a form by which they are taken up by the cells so that therapeutically effective levels of the inhibitory nucleic acid molecules are contained within the cells.

Introduction of nucleic acids into cells may be accomplished using any number of methods available in the art. For example, transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, an inhibitory nucleic acid or miRNA (or a precursor to the miRNA) as described can be cloned into a retroviral vector where expression can be driven from its endogenous promoter, from the retroviral long terminal repeat, or from a promoter specific for a target cell type of interest. In some embodiments, the target cell type of interest is an endothelial cell. Other viral vectors that can be used to introduce nucleic acids into cells include, but are not limited to, vaccinia virus, bovine papilloma virus, or herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In some embodiments, a viral vector is used to administer a polynucleotide encoding inhibitory nucleic acid molecules that inhibit expression of ACSS2.

Non-viral approaches can also be employed for the introduction of the therapeutic to a cell of a patient requiring treatment of atherosclerosis. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In some embodiments, the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA and RNA into a cell. Transplantation of polynucleotide encoding inhibitory nucleic acid molecules into the affected tissues of a patient can also be accomplished by transferring a polynucleotide encoding the inhibitory nucleic acid into a cultivatable cell type ex vivo (e.g., an autologous or heterologous primary cell or progeny thereof), after which the cell (or its descendants) are injected into a targeted tissue. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In some embodiments, the therapeutic polynucleotide is selectively targeted to an endothelial cell. In some other embodiments, the therapeutic polynucleotide is expressed in an endothelial cell using a lentiviral vector. In still other embodiments, the therapeutic polynucleotide is administered intravenously. In some embodiments, the therapeutic polynucleotide contains one or more chemical modifications that reduce immunostimulation, enhance serum stability, increase specificity, and/or improve activity, while still retaining silencing activity. Such chemical modifications are described in, for example, Foster et al., RNA. 2012 March; 18(3): 557-568. In some embodiments, the therapeutic polynucleotide contains one or more chemical modifications to prevent degradation, as described in Chen et al., Cell Reports 2012; 2(6)1684-1696.

In a particular embodiment, the therapeutic polynucleotide is selectively delivered to endothelial cells using nanoparticles formulated for selective targeting to endothelial cells, such as a 7C1 nanoparticle. Selective targeting or expression of polynucleotides to an endothelial cell is described in, for example, Dahlman et al., Nat Nanotechnol. 2014 August; 9(8): 648-655.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The results of the experiments are now described.

### Example 1: Characterization of TGFβ Signaling During EntMT

A number of studies have established endothelial-to-mesenchymal transition (EndMT) as the key pathological event responsible for chronic vascular inflammation. EndMT underlies and drivers a wide spectrum of diseases from atherosclerotic cardiovascular diseases (MI, strokes), pulmonary hypertension, vascular graft failure, and numerous fibrotic states. The key to EndMT development is activation of endothelial TGFβ signaling that transforms this cell type into mesenchymal-like cells. In order to better understand the activation and regulation of TGFβ signaling, studies were conducted which examined the metabolic basis of TGFβ signaling with the aim of identifying novel therapeutic targets.

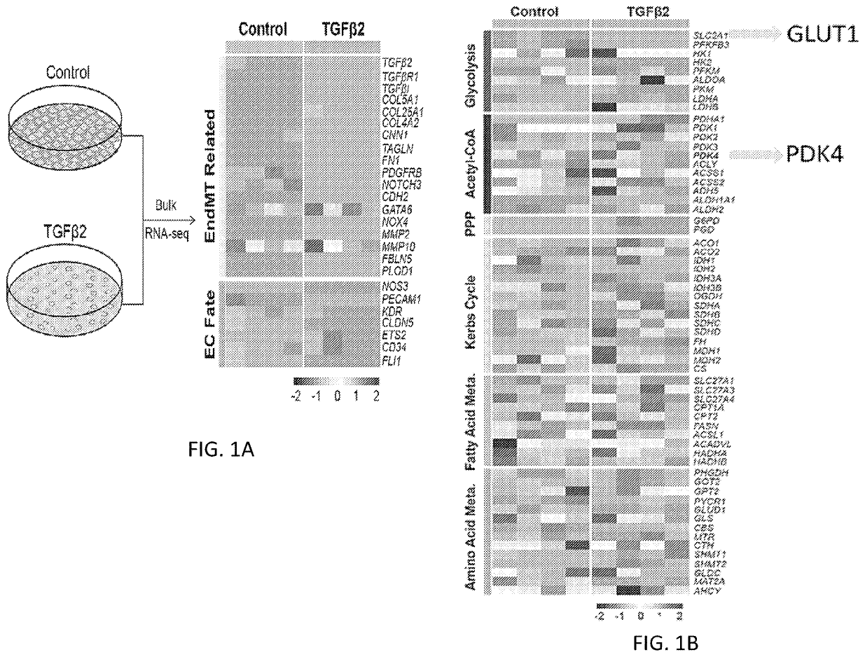

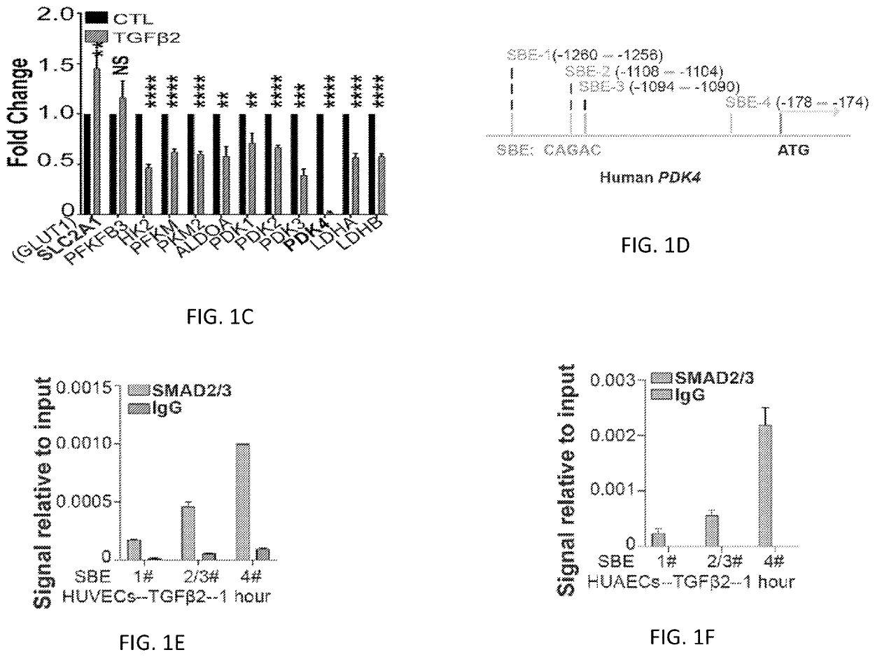

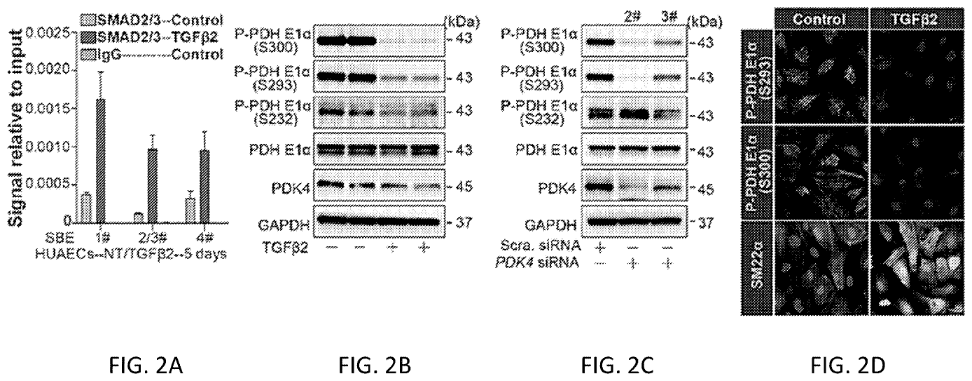

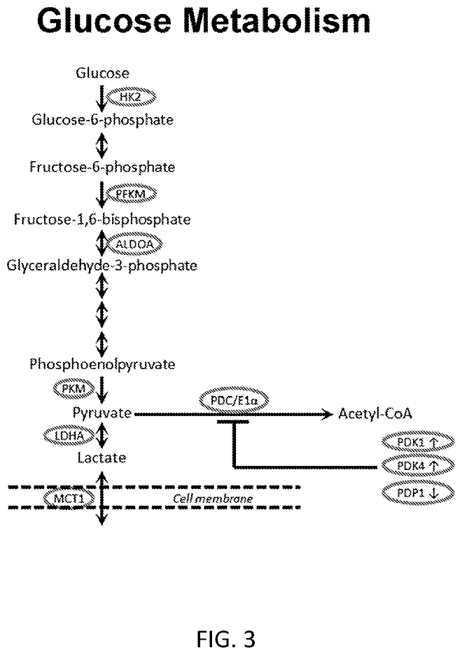

TGFβ treatment of endothelial cells (ECs) growing in culture results in number of gene expression changes which have become well-known as markers of the EndMT process (FIG. 1A). RNAseq analysis of metabolic gene expression showed a large increase in expression of a glucose transporter Glu-1 (SLC2A1) and a profound decrease in expression of PDK4 (FIGS. 1B, 1C). Human PDK4 possesses a Smad-2/3 binding site and ChiP analysis demonstrated the expected increase in Smad-2/3 binding to PDK4 promoter after initiation of TGFβ treatment (FIGS. 1D-IF). The primary result of this inhibition of PDK4 expression was an increase in activity of the PDH complex (PDC) due to dephosphorylation of PDHE1α(FIG. 2). The result of this signaling activity is an expected increase in acetyl CoA levels (FIG. 3).

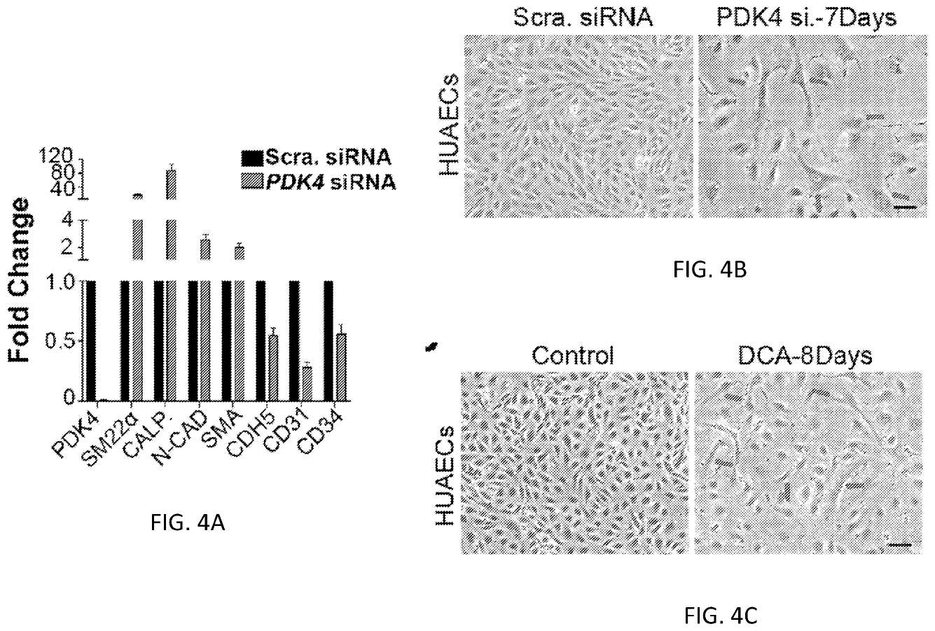

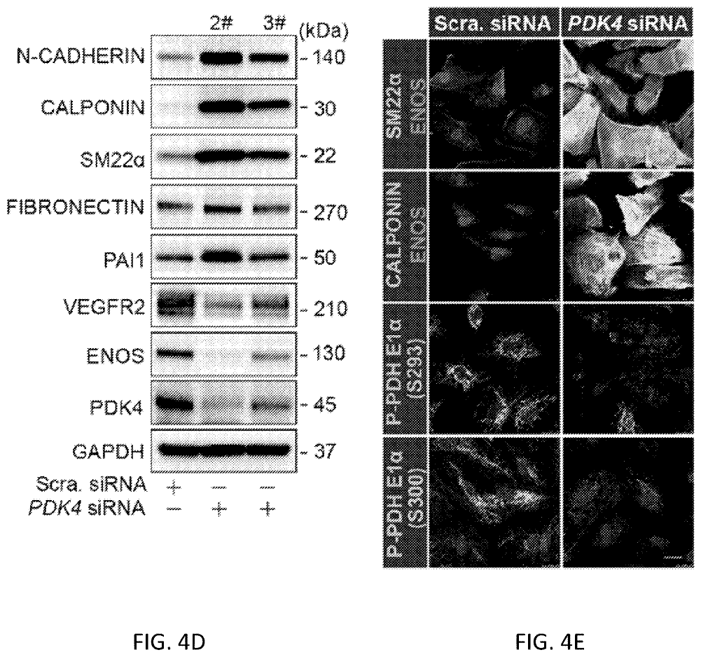

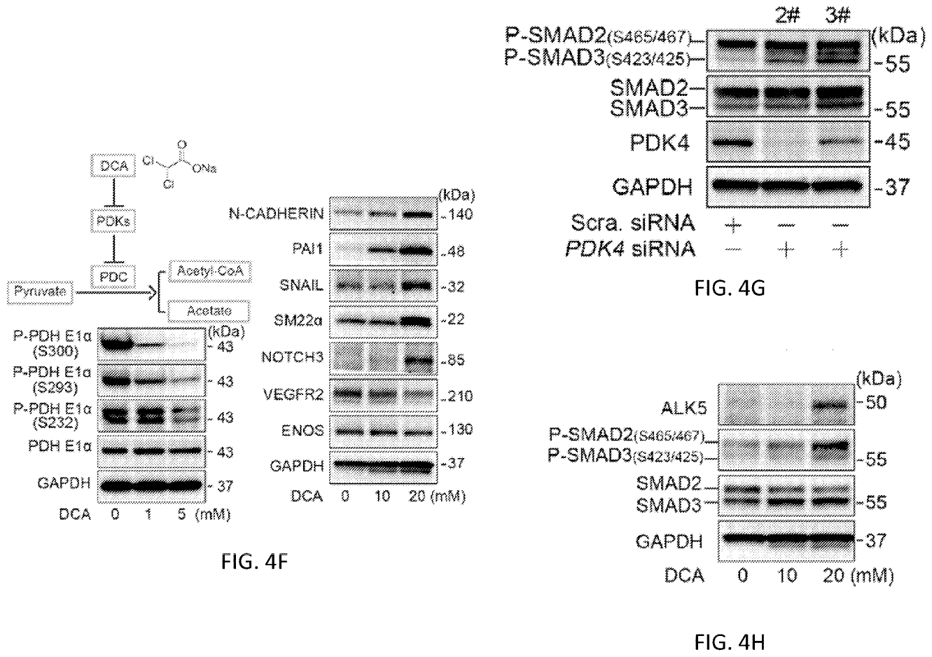

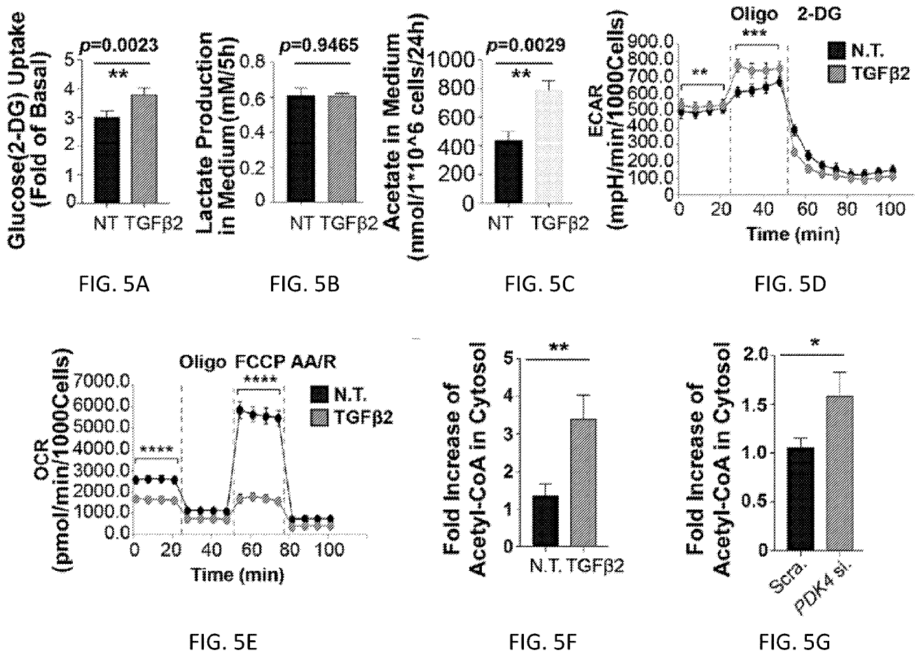

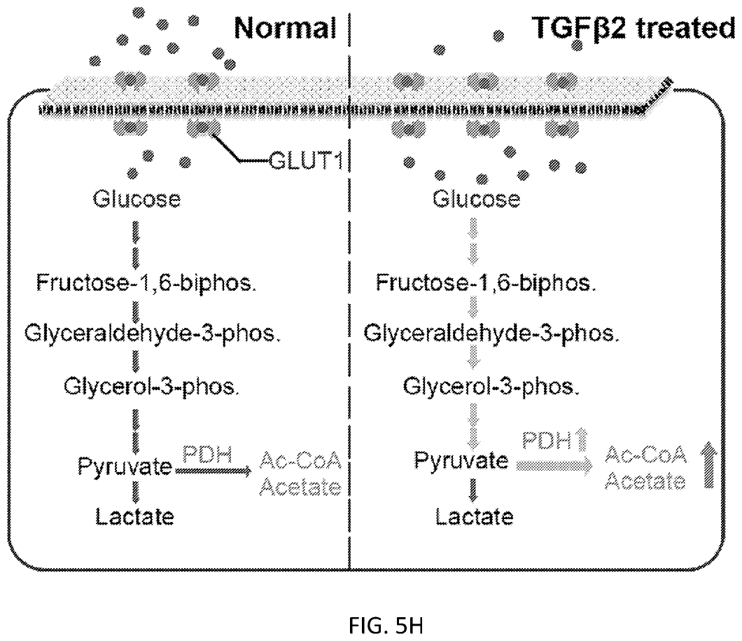

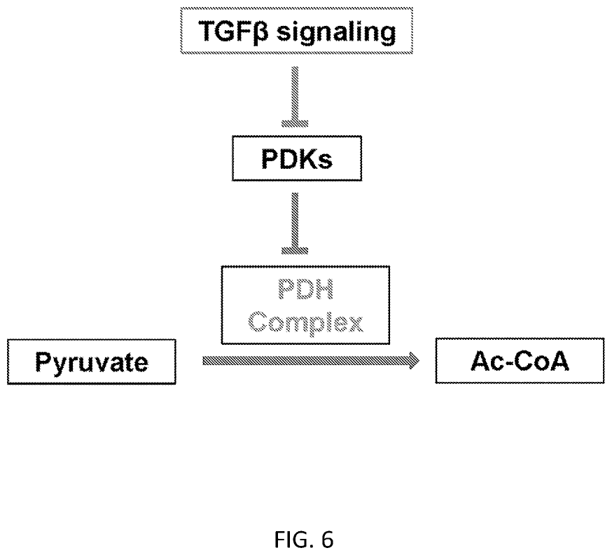

In order to determine if PDK4 deficiency by itself can induce EndMT, its expression was knocked-down in endothelial cells (FIG. 4A), which were then assessed for molecular and phenotypic changes. Seven days later endothelial cells were observed to have assumed a more mesenchymal appearance (FIGS. 4B, 4C) and demonstrated increased expression of mesenchymal markers while endothelial cell markers declined (FIGS. 4D, 4E). Treatment with the PDK inhibitor dichloroacetic acid (DCA) had a similar effect (FIG. 4F). An increase in phosphorylated Smad-2/3 expression was also observed, which is consistent with increased TGFβ signaling activity. Likewise, further analysis demonstrated that TGFβ treatment of endothelial cells increased levels production of acetyl-CoA (FIG. 5). Importantly, PDK4 knockdown resulted in acetyl-CoA increases that were comparable to those induced by TGFβ treatment alone (FIGS. 5G,5H). Taken together, and without wishing to be bound by theory, these data demonstrate that TGFβ promotes acetyl-CoA production by inhibiting PDK4 that, in turn, leads to activation of PDH complex (PDC) (FIG. 6).

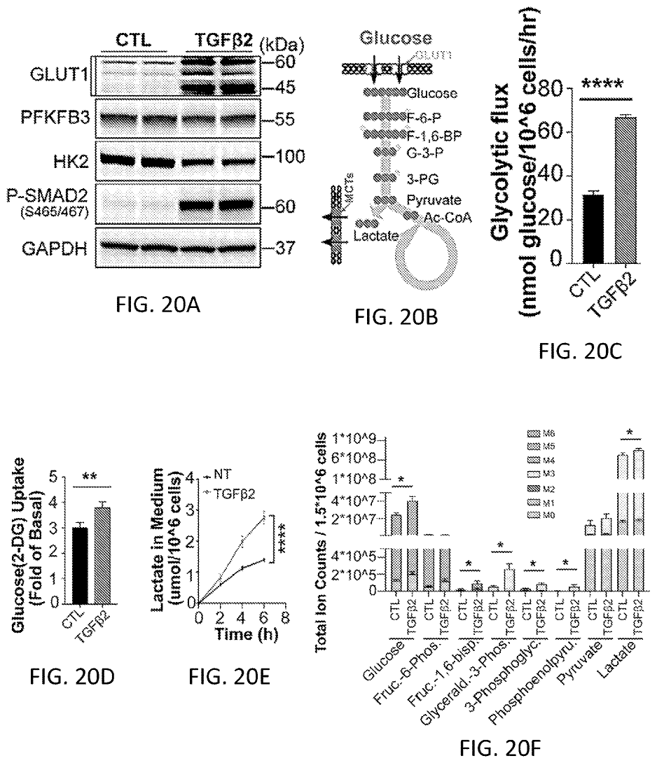

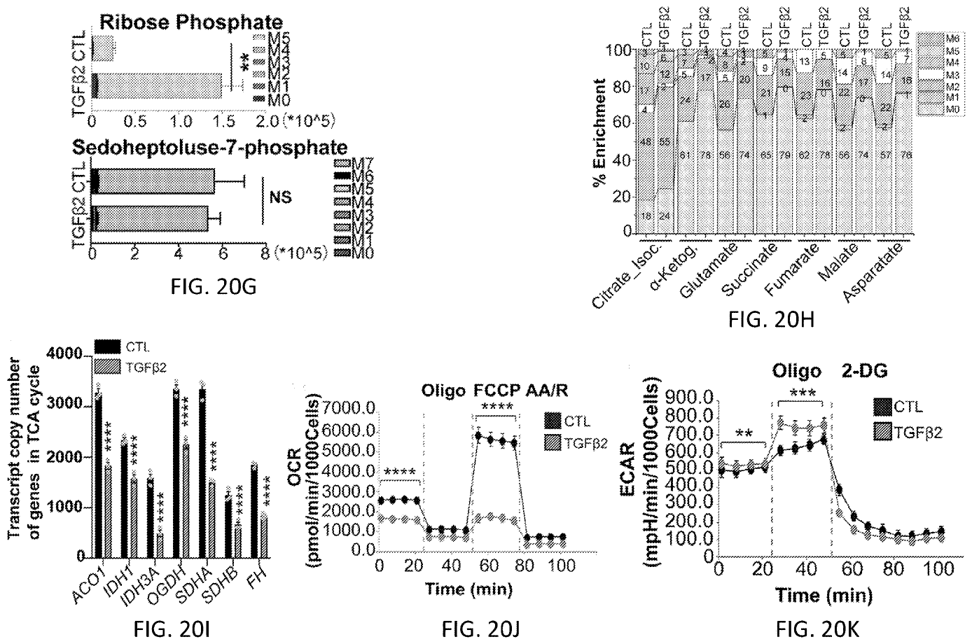

In agreement with increased GLUT1 protein levels (FIG. 20A), there was a significant increase in the glycolytic flux comprises three subunits. The rate-limiting E1 subunit cata-(FIGS. 20B and 20C) and glucose uptake (FIG. 20D), as well the decarboxylation and dehydrogenation of pyruvate. The as increased lactate secretion (FIG. 20E). To further analyze the effect of these changes on endothelial metabolism, HUAECS were treated with 13C-glusose after 7 days of TGFβ stimulation. This revealed a significant increase in the level of key intermediaries, as well as 13C-glucose contribution to their biosynthesis (FIG. 20F).

Further, a reduction in expression of PPP enzymes was observed, with no significant changes in glucose-derived sedoheptulose-7-phosphate levels but a large increase in glucose-derived ribose-5-phosphate (FIG. 20G), likely driven by increased glucose uptake. Tracing of 13C-glucose contribution to the TCA cycle showed that, although its incorporation into citrate/isocitrate was only mildly reduced, its contribution to a-ketoglutarate and subsequent TCA metabolites production decreased significantly, implying either a block at the level of isocitrate dehydrogenase (IDH) or increased production of a-ketoglutarate and subsequent TCA metabolites production decreased significantly (FIG. 20H), implying either a block at the level of isocitrate dehydrogenase (IDH) (FIG. 20I) or increased production of a-ketogluarate from glutamine. In agreement with a block in TCA metabolism and a rise in glycolysis and lactate secretion, oxygen consumption rate (OCR) was profoundly reduced (FIG. 20J) and extracellular acidification rate (ECAR) was increased (FIG. 20K) TGFβ-treated cells.

### Example 2: Regulation of Ac-CoA Production by ACSS2

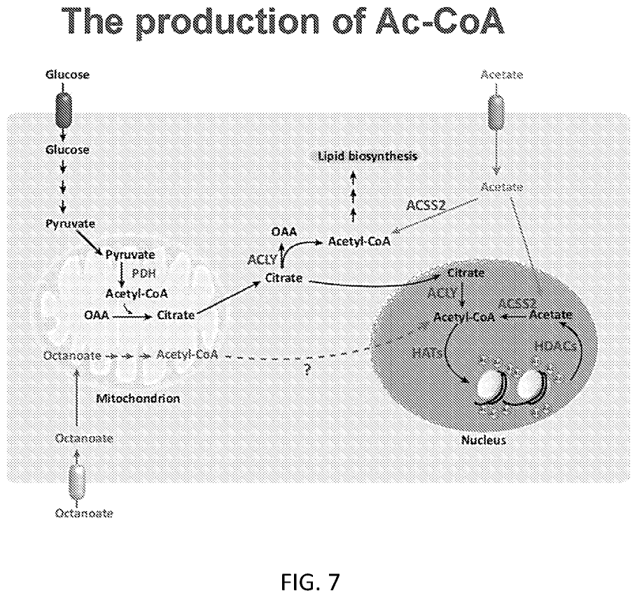

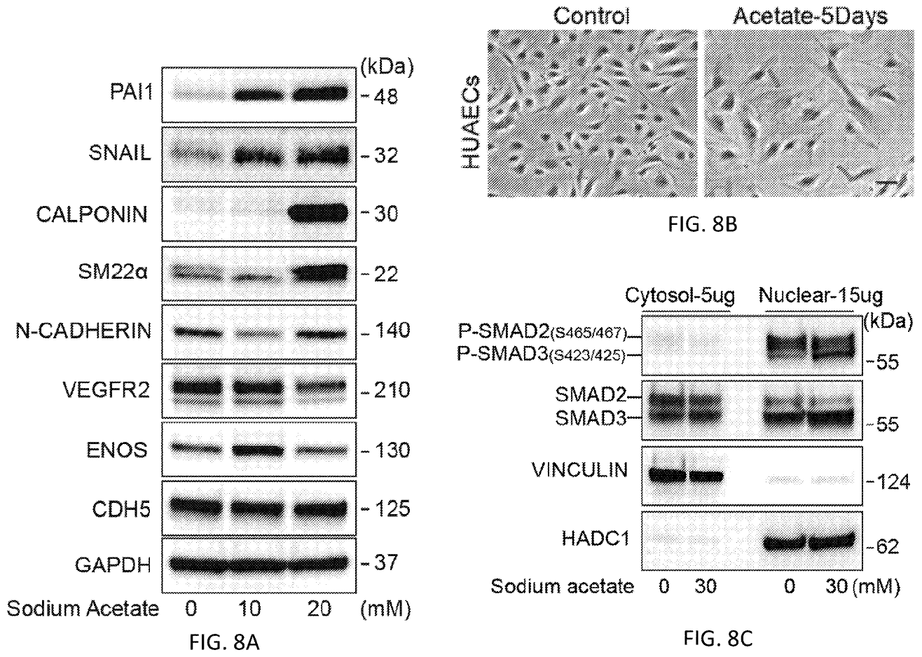

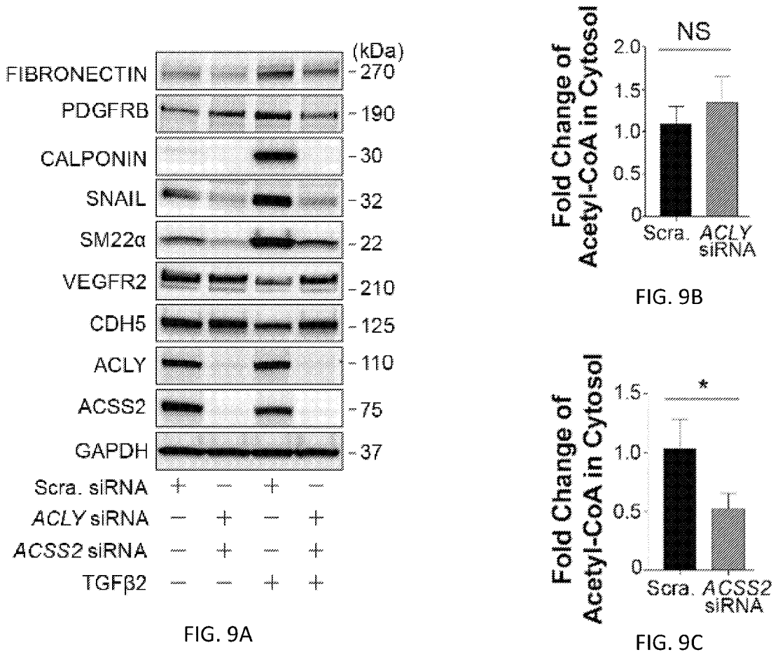

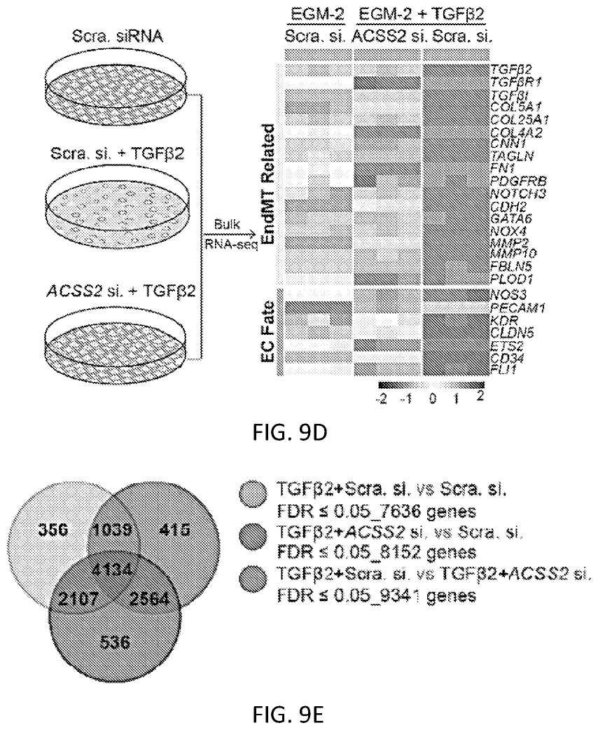

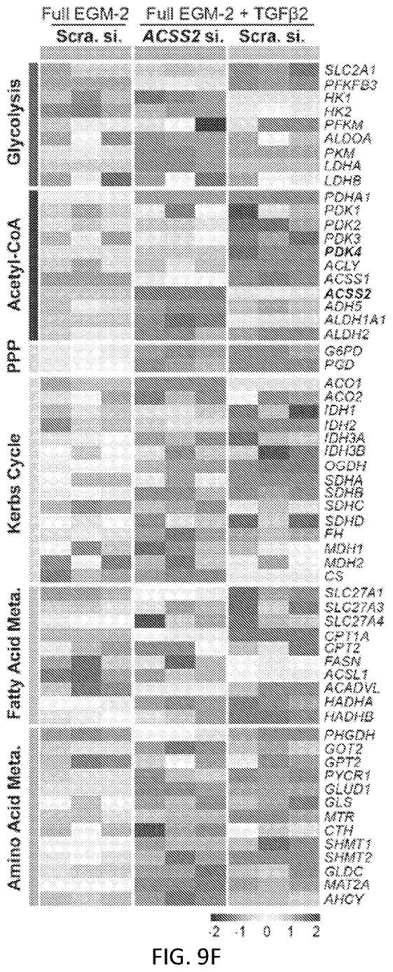

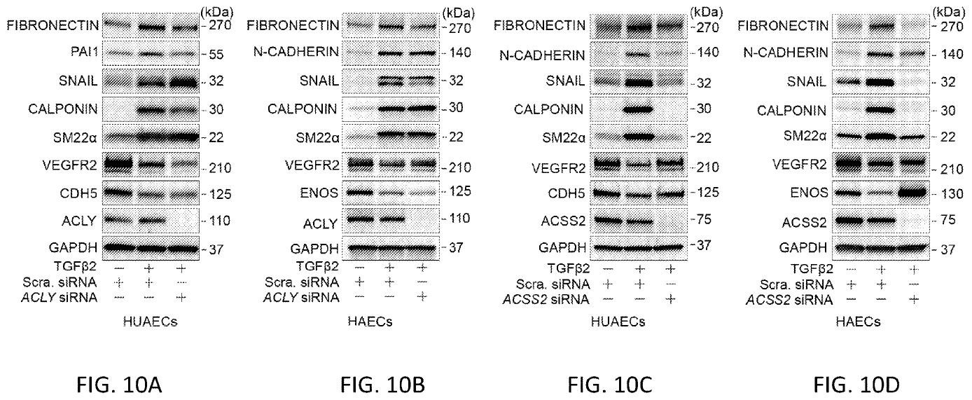

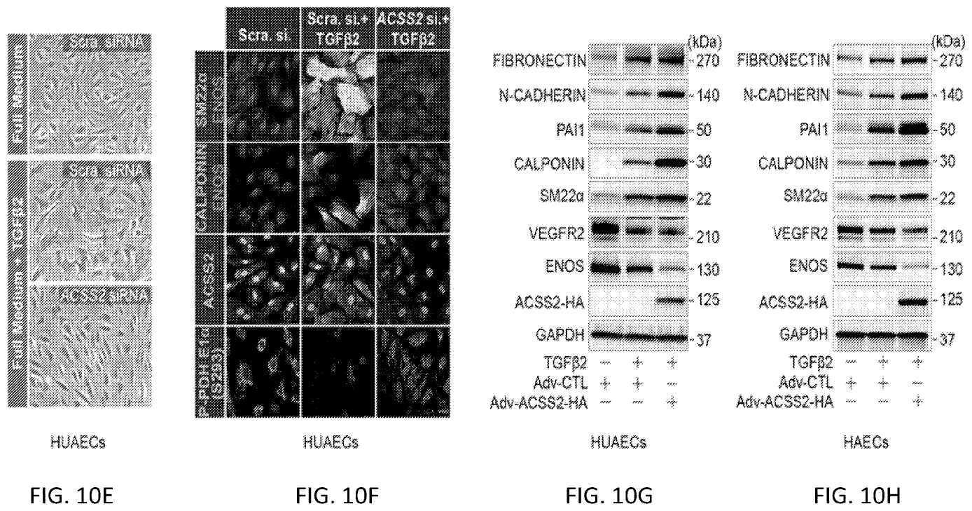

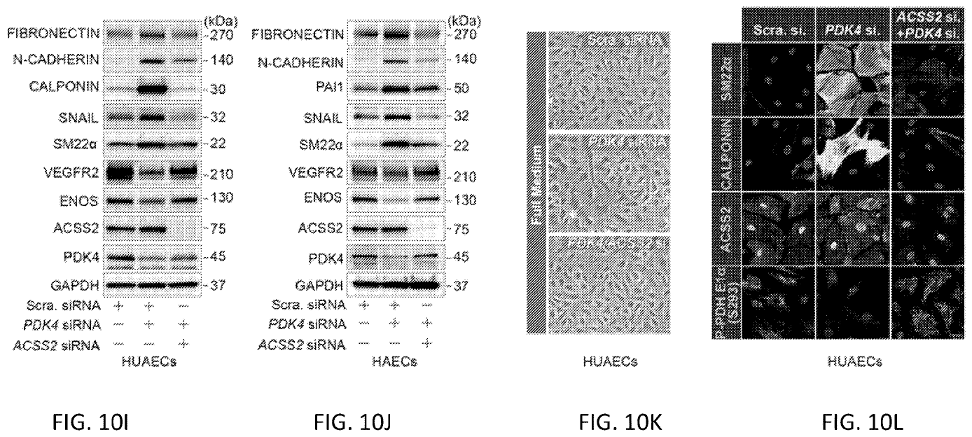

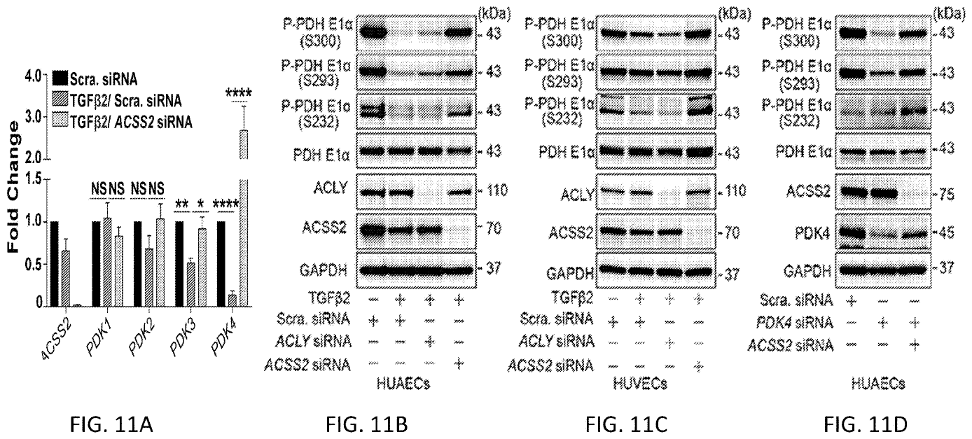

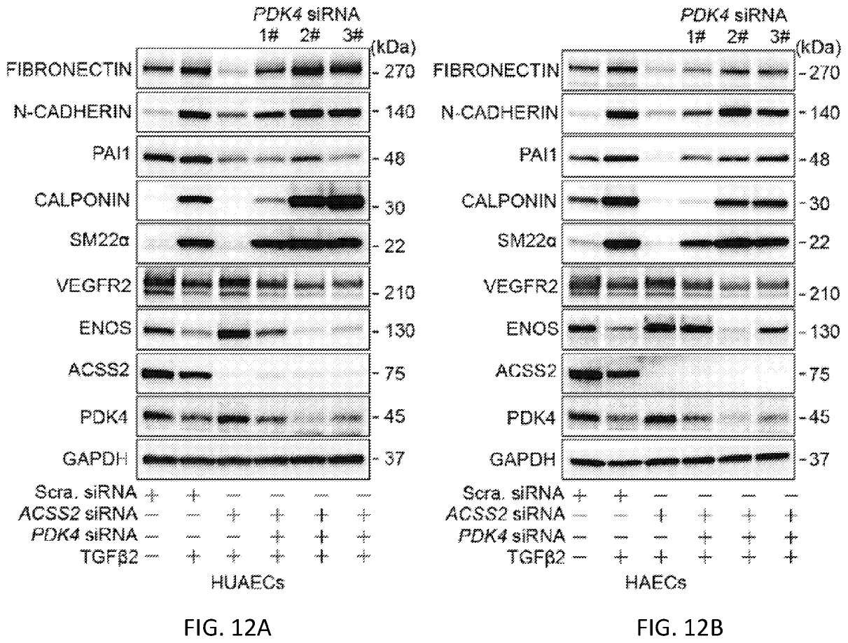

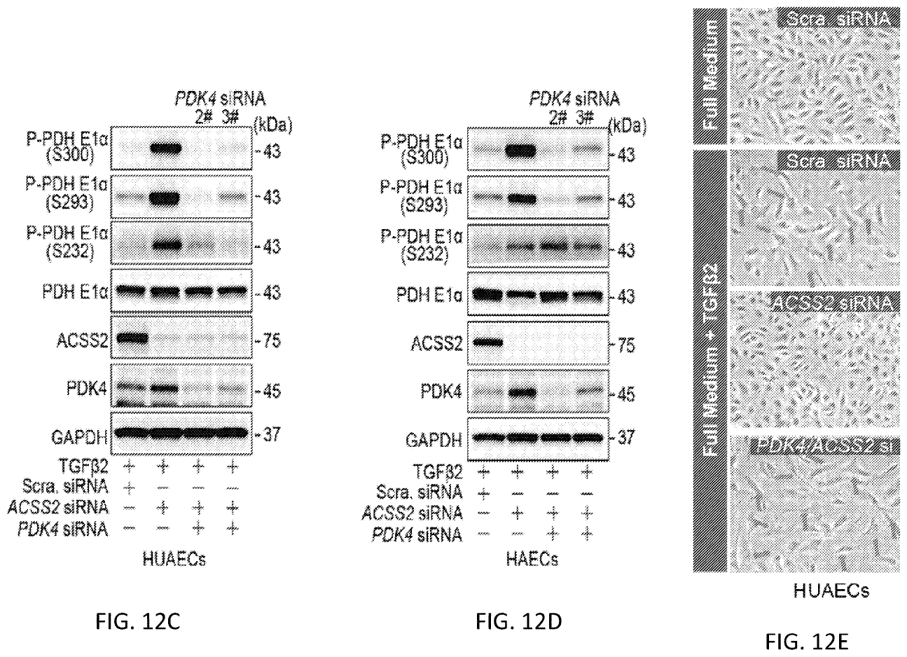

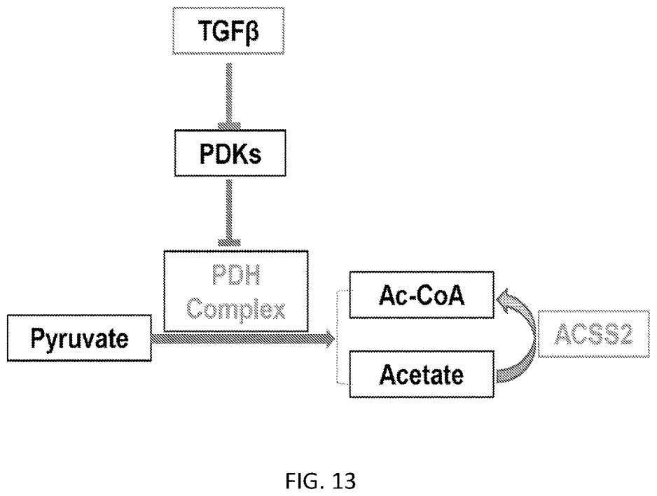

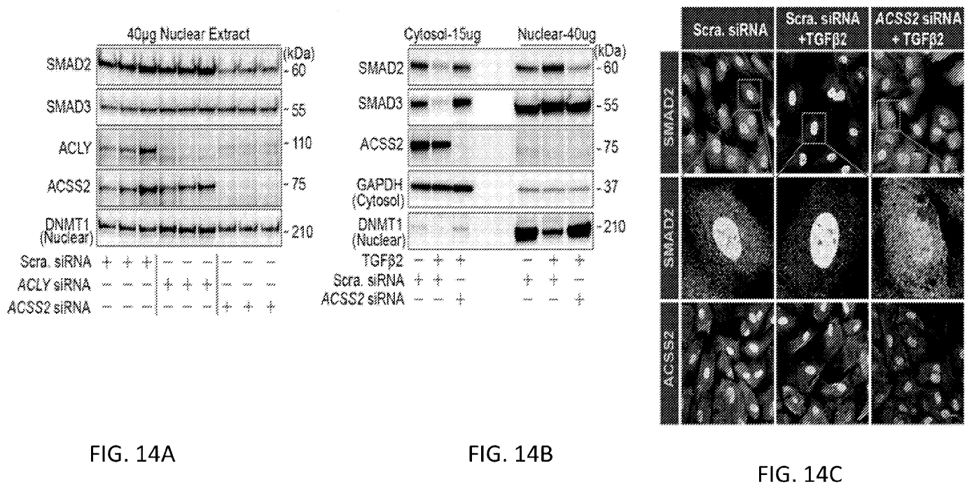

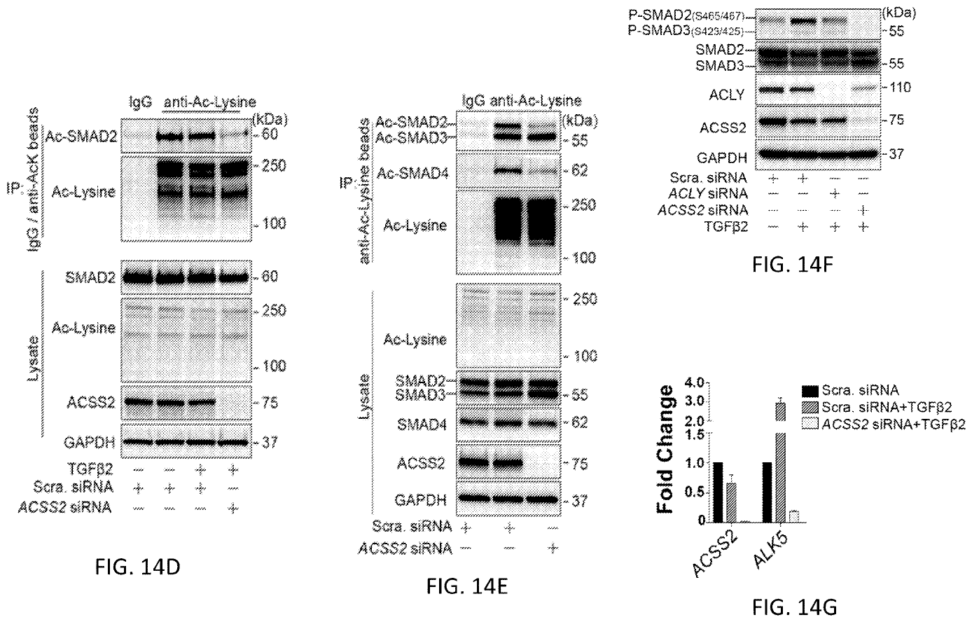

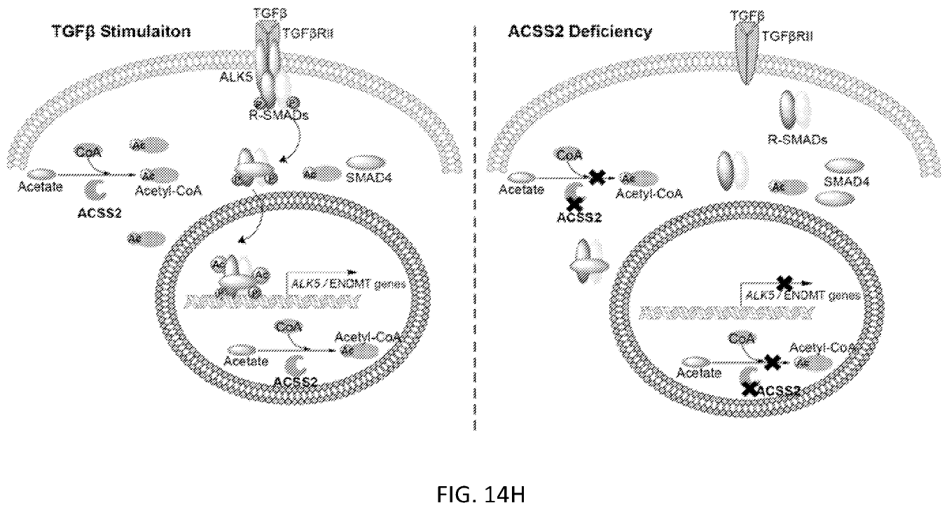

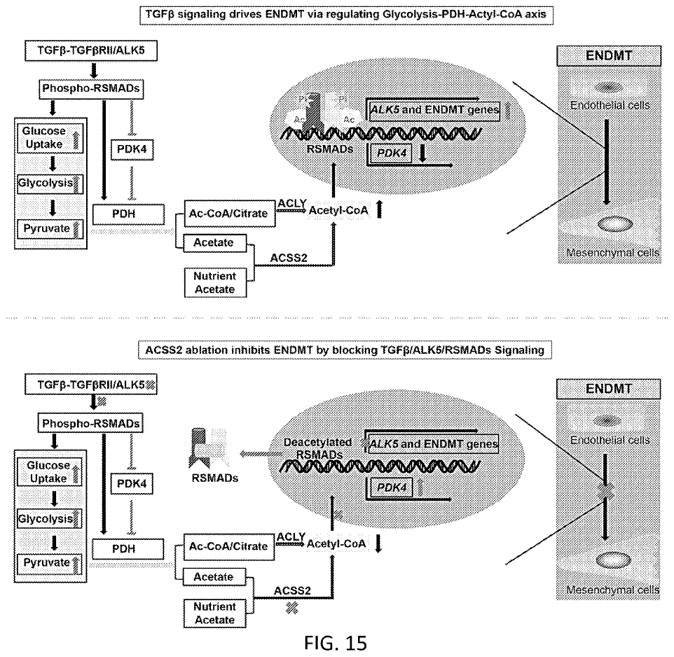

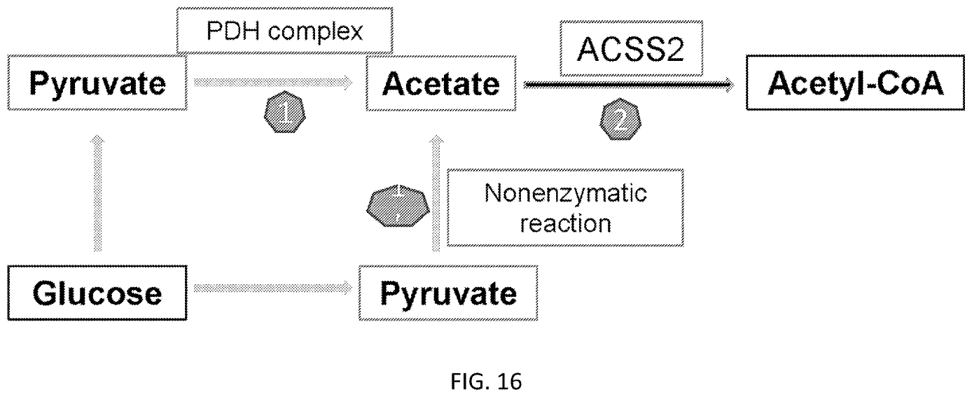

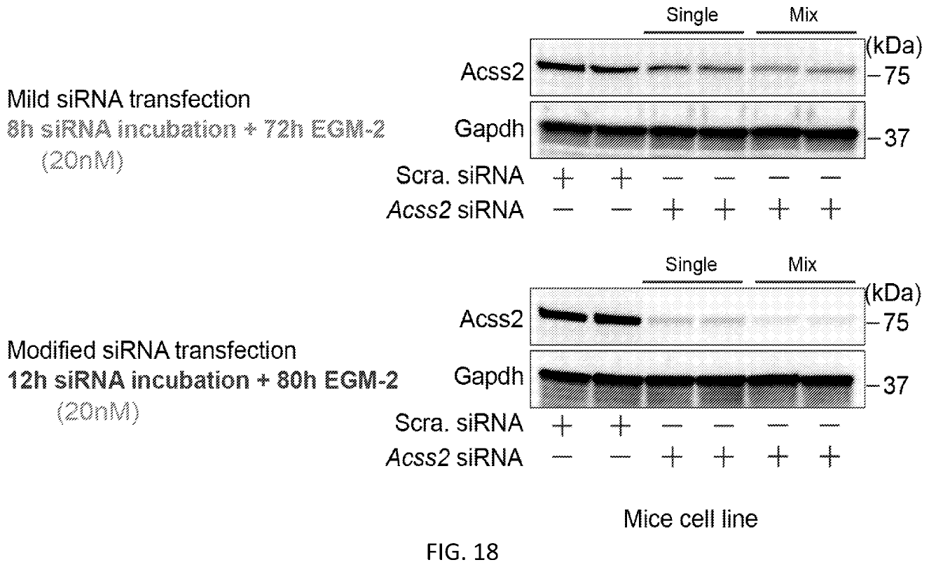

The two major sources of acetyl-CoA are acetate and glucose via pyruvate (FIG. 7). Studies were then conducted in order to better characterize which source of acetyl-CoA production is involved in the observed signaling processes. Previous experiments had noted that increased amounts of acetate in the cell culture medium promoted EndMT (FIG. 8). The two principal enzymes responsible for Ac-CoA production are ATP Citrate Lyase (ACLY) and Acyl-CoA Synthetase Short Chain Family Member 2 (ACSS2). In order to directly assess the role of ACSS2, a series of siRNAs were constructed targeting this gene (see Table 1), which were verified to be capable of knocking down ACSS2 expression in mouse endothelial cells (FIG. 18). A knockdown of ACSS2 but not that of ACLY, reduced acetyl-CoA levels in endothelial cells and reversed the consequences of TGFβ signaling (FIGS. 8-9). Knock-down of ACSS2 expression also rescued EndMT induced by the knock-down of PDK4 expression (FIG. 10). On the molecular level, ACSS2 knock-down increased PDHE1αphosphorylation by restoring PDK4 expression (FIG. 11). In agreement with this paradigm, PDk4 knock-down prevented ACSS2 knock-down from rescuing EndMT (FIG. 12). Results of these studies are summarized in FIGS. 14H-16. Without wishing to be bound by theory, these data demonstrate a key role for ACSS2 acetyl-CoA producing activity in the process of EndMT, and suggest that the ACSS2-targeting strategies of the current invention can be used to block EndMT and the effects of TGFβ. The importance of these processes in chronic vascular inflammation further highlights the utility of targeting ACSS2 for the treatment of these diseases.

### Example 3: Inhibition of ACSS2 Expression

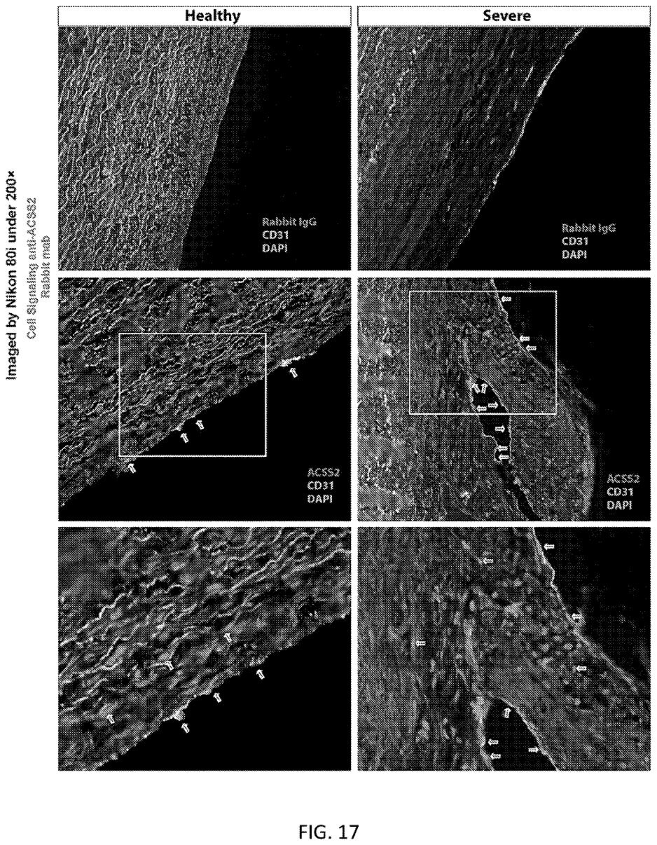

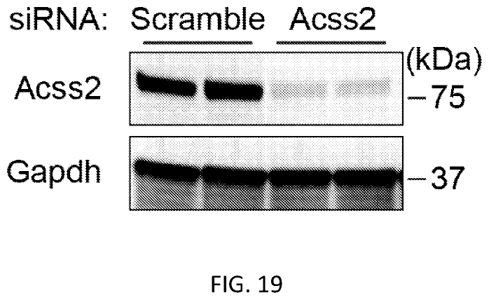

The studies of the current disclosure having demonstrated the role of ACSS2 in the Acetate--ACSS2--Acetyl-CoA axis that drives the process of EndMT, a series of studies was then undertaken in order to correlate the expression of ACSS2 to EndMT-associated disease states in human endothelial tissue. To this end, tissue specimens from a patient suffering from severe atherosclerosis and a normal control were obtained for histological analysis. After fixing, embedding in paraffin, and sectioning the tissue onto slides, the specimens were stained for expression of ACSS2 using fluorescent-labeled antibodies. CD31 was also stained in order to visualize endothelial cells. The results of these studies demonstrated a dramatic increase in ACSS2 expression in the endothelial tissue from atherosclerotic patients as compared to the normal control (FIG. 17). Without wishing to be bound by theory, these data suggested that ACSS2 is not only a marker of atherosclerotic tissue, but also that targeting its expression in vivo could serve as a treatment for this disease. In order to demonstrate that ACSS2 expression could be targeted by siRNA knockdown in human cells, studies were conducted using primary cultures of human endothelial cells. Analysis of ACSS2 expression by Western blot revealed dramatic knockdown in cells treated with the targeting siRNAs but not the scramble controls (FIG. 19) demonstrating the feasibility of this strategy in humans.

### Example 4: TGF-β Regulates PDK4 Expression and Ac-CoA Synthesis

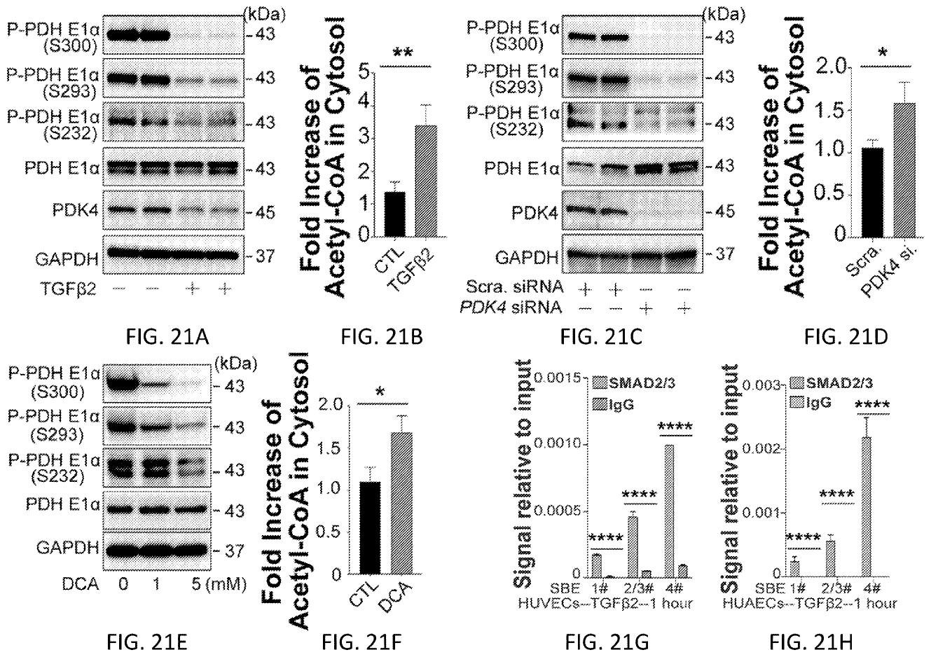

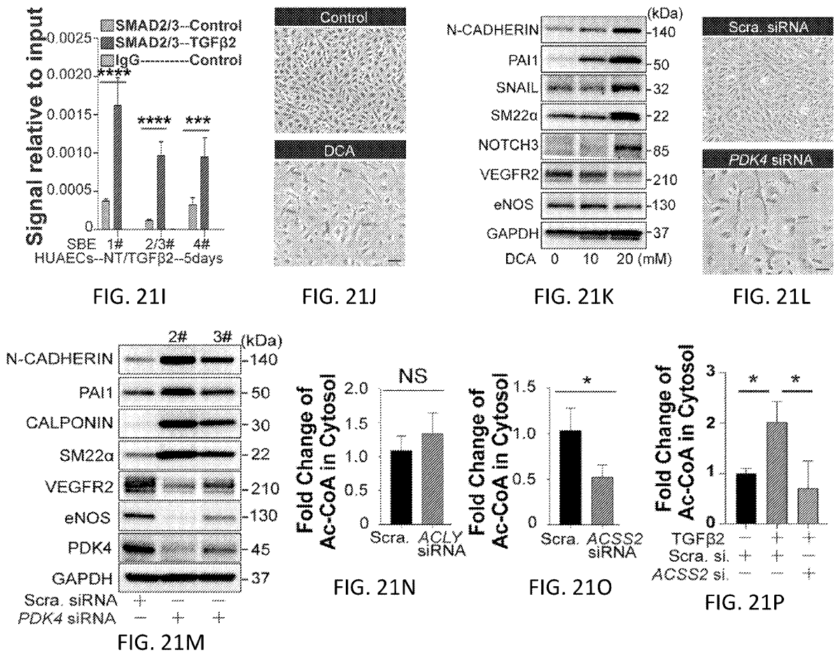

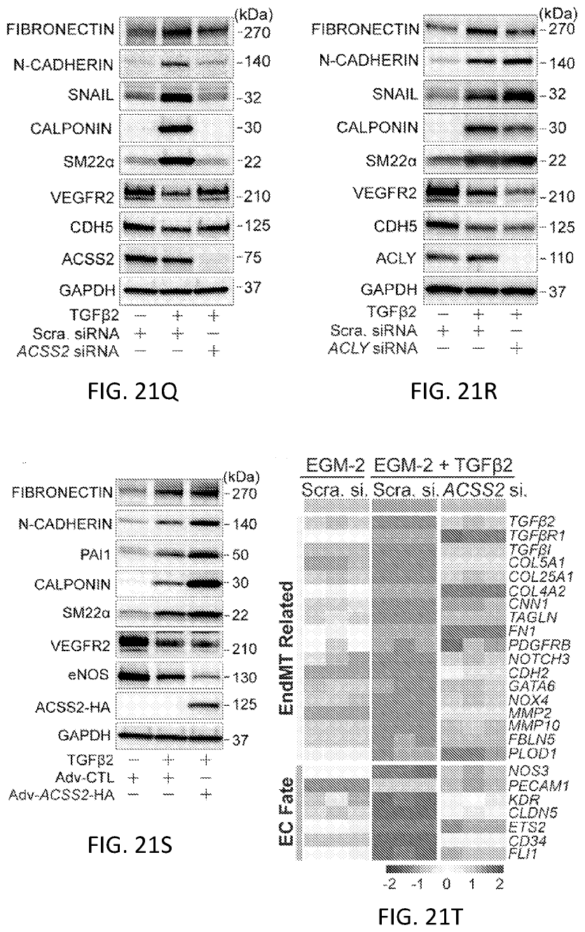

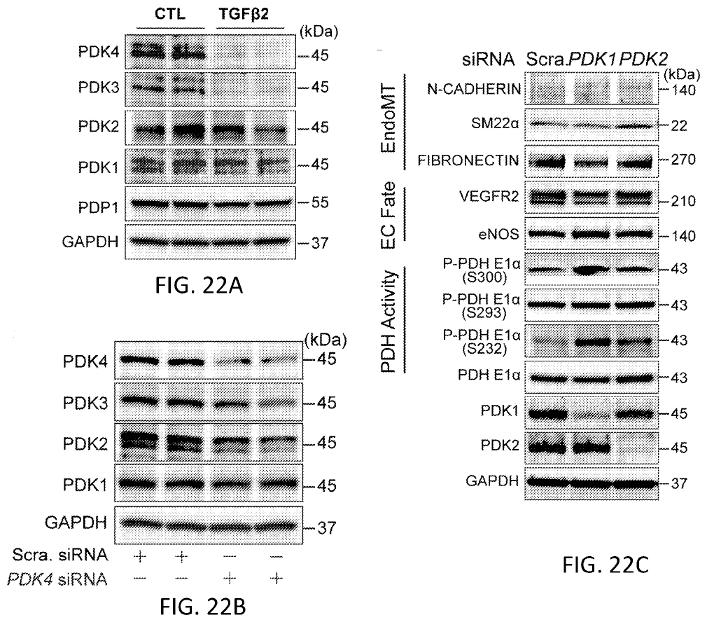

One surprising effect of TGFβ stimulation was a near-complete reduction in expression of PDK4, a key inhibitor of PDH-dependent Ac-COA biosynthesis (FIGS. 1C and 21A). In agreement with the PDH-inhibiting role of PDK4, the TGFβ-induced reduction in PDK4 expression was associated with a near-complete loss of PDH phosphorylation (FIG. 2A) and a significant increase in cellular Ac-COA levels (FIG. 21B). Treatment of HUAECs with siRNAs to PDK4 led to a similar loss of PDH phosphorylation and an increase in Ac-COA levels (FIGS. 21C and 21D), confirming that the loss of PDK4 is sufficient to link TGFβ stimulation with increased Ac-COA production. Furthermore, inhibition of PDK4 activity with the pan-PDK inhibitor dichloroacetate (DCA) similarly resulted in decreased PDH-E1a phosphorylation (FIG. 21E) and increased Ac-COA production (FIG. 21F).

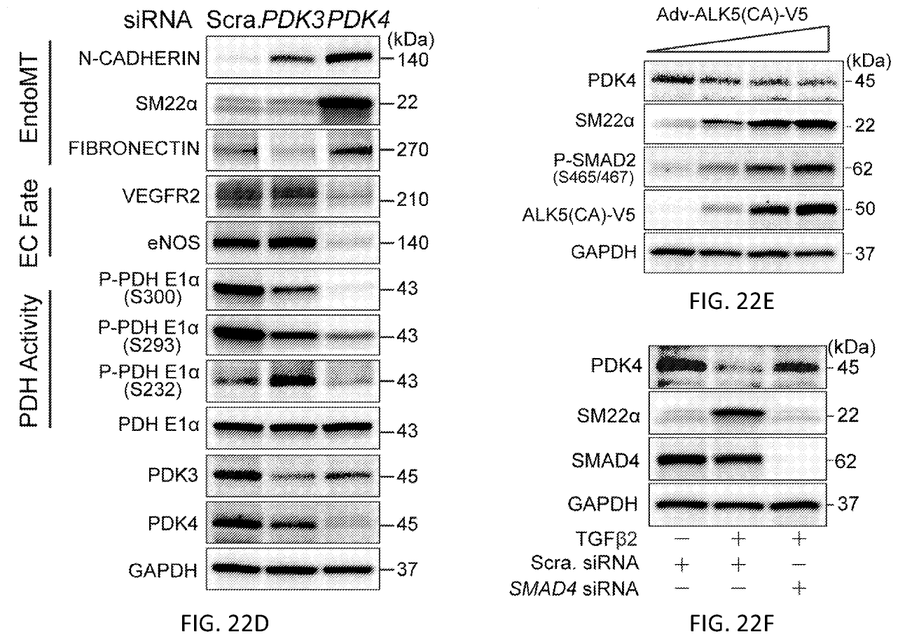

To confirm that PDK4 is the primary PDK affected by TGFβ treatment, protein levels of all PDK isoforms were measured after 5 days of TGFβ stimulation. In agreement with RNA-seq data, western blotting showed a profound reduction in PDK3 and PDK4 expression, whereas PDK1 and 2 levels were not affected (FIG. 22A). Similar changes were observed after PDK4 siRNA mediated knockdown (FIG. 22B). To further assess the functional role of PDK isoforms, each isoform was knocked down using siRNA. Although knockdowns of either PDK1 or PDK2 had no effect on expression of endothelial fate and EndMT markers (FIG. 22C), a knockdown of PDK4 induced a profound reduction in expression of endothelial fate genes and a strong EndMT marker induction, whereas PDK3 knockdown had only mild effects (FIG. 22D).

To understand how TGFβ stimulation leads to a reduction in PDK4 expression, in silico analysis of the human PDK4 promoter was performed. This identified four potential SMAD-binding elements (SBEs, FIG. 1D). ChIP-PCR testing of TGFβ-treated HUAECs showed a rapid increase in Smad-2/3 binding to three of the four SBEs starting at 1 h and to all four SBEs after 5 days (FIGS. 21G-21I), suggesting a direct regulation of PDK4 expression by SMADs. Furthermore, adenoviral overexpression of a constitutively active form of ALK5 (ALK5-CA) that leads to increased TGFβ signaling as measured by increased SMAD-2 phosphorylation, resulted in decreased PDK4 expression (FIG. 22E). Finally, inhibition of TGFβ signaling by SMAD4 siRNA interference restored PDK4 protein expression even in the presence of TGFβ (FIG. 22F). These results demonstrate that canonical TGFβ/ALK5/R-SMADs signaling plays a critical role in regulating PDK4 expression.

### Example 5: Endothelial Ac-COA is Largely Derived from Acetate, and Acetate Drives EndMT

Because ACSS2 mediates Ac-CoA synthesis from acetate, these data implicated acetate as a central driver of EC metabolism and of EndMT and prompted further studies to probe more deeply into endothelial acetate metabolism. ¹³C-labeled substrate studies were used to investigate if ECs can in fact generate endogenous acetate and, if so, from what substrates and to what end. It was found that in the presence of physiological levels of ambient acetate (100 mM), ECs both consume and secrete substantial amounts of acetate, i.e., labeled acetate in the media was consumed while simultaneously unlabeled acetate was secreted into the media (FIGS. 3A and 3B).

### Example 6: Increase in Endothelial Ac-COA Induces EndMT in ACSS2-Dependent Manner

Because inhibition of PDK4 activity or expression even in the absence of TGFβ leads to an increase in Ac-COA levels, studies were then conducted to determine if that increase is sufficient for induction of EndMT. Seven days after initiation of DCA treatment, HUAECs acquired a distinct spindleform morphology, losing their normal cobblestone appearance (FIG. 21J). Analysis of gene expression documented increased expression of a range of EndMT markers including N-Cadherin, SM22a, and PAI1 (FIG. 21K). Similarly to DCA-induced inhibition of PDK activity, siRNA-mediated PDK4 knockdown also resulted in EndMT induction, verified both morphologically and by western blotting (FIGS. 21L and 21M).

These results link the increase in endothelial Ac-COA levels to EndMT induction, in a manner dependent on PDH activity. Ac-COA generated by PDH, which is located in the mitochondrial matrix, generally has one of two fates: incorporation into, and oxidation by, the TCA cycle, or export from the mitochondria via the citrate shuttle, a process dependent on regeneration of Ac-COA from citrate in the cytoplasm by the enzyme ACLY. To test if ACLY is indeed involved in Ac-COA generation in the endothelium, expression of ACLY was knocked down in HUAECs. Surprisingly, ACLY knockdown had no effect on baseline endothelial Ac-CoA level (FIG. 21N), indicating that the bulk of Ac-COA is produced by other means. Ac-COA can be alternatively generated by conjugation of CoA directly with acetate, a process requiring the enzyme ACCS2.19 Consistent with the use of this alternative means of CoA generation, knockdown of ACSS2 significantly reduced baseline endothelial Ac-COA (FIG. 21O). In agreement with the observed reduction in baseline Ac-COA, a knockdown of ACSS2 also markedly reduced the extent of TGFβ-induced increase in Ac-COA (FIG. 21P) and EndMT (FIG. 21Q), whereas ACLY KD had no effect (FIG. 21R). Conversely, increased expression of ACSS2, achieved by adenoviral-mediated transduction of HUAECs, potentiated the extent of TGFβ-induced EndMT (FIG. 21S). Importantly, in addition to reducing EndMT, ACSS2 knockdown also restored expression of endothelial fate genes (FIG. 21T). Similar results were obtained in HAECs (FIGS. 10B-10D).

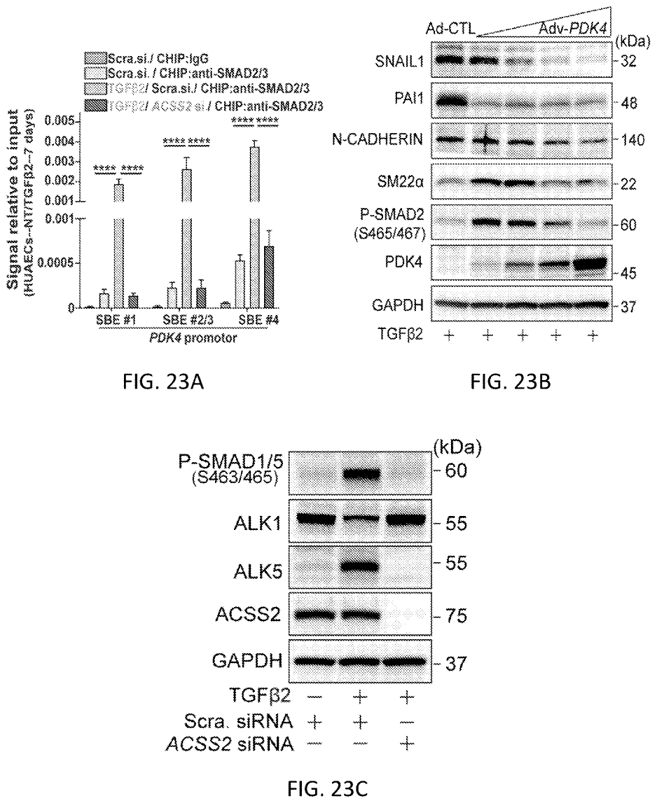

Finally, ACSS2 knockdown in HUAECs reversed TGFb-induced increased expression of GLUT1 and restored normal expression of various metabolic enzymes among others (FIG. 9F). In particular, PDK4 expression returned to normal levels (FIG. 11A). In agreement with these changes in PDK4 expression, decreased Smad-2/3 binding to PDK4 SBEs was observed after ACSS2 knockdown (FIG. 23A). To test whether EndMT induction by TGFβ stimulation could be rescued by restoring PDK4 levels, adenoviral PDK4 overexpression was employed after 3 days of TGFβ stimulation. This resulted in a PDK4 overexpression level-dependent decrease in EndMT markers (FIG. 23B). Of note, a decrease of ALK1 induced by TGFβ stimulation was reversed by ACSS2 knockdown in the presence of TGFβ stimulation (FIG. 23C).

### Example 7: Endothelial Ac-COA is Largely Derived from Acetate, and Acetate Drives EndMT

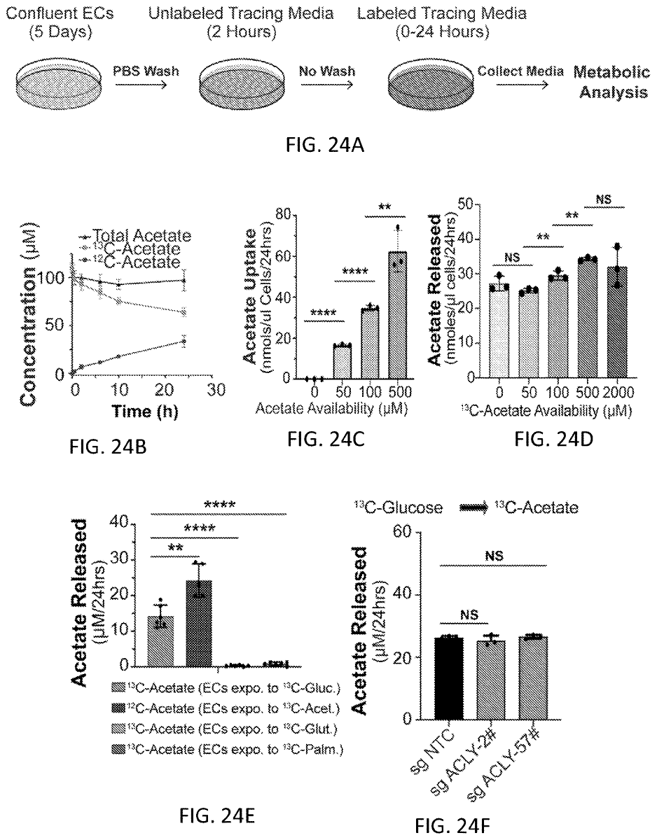

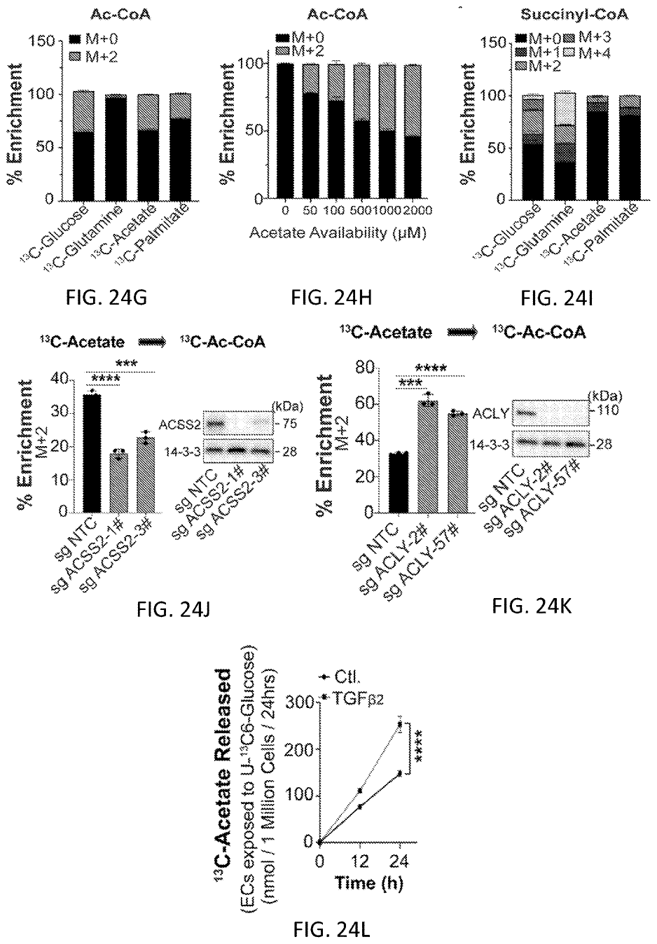

Because ACSS2 mediates Ac-CoA synthesis from acetate, these data implicated acetate as a central driver of EC metabolism and of EndMT and prompted us to probe more deeply into endothelial acetate metabolism. ¹³C-labeled substrate studies were used to investigate if ECs can in fact generate endogenous acetate and, if so, from what substrates and to what end. It was found that in the presence of physiological levels of ambient acetate (100 mM), ECs both consume and secrete substantial amounts of acetate, i.e., labeled acetate in the media was consumed while simultaneously unlabeled acetate was secreted into the media (FIGS. 24A and 24B).

Interestingly, changing the amount of available acetate in the media changed the rate of uptake, but the rate of production remained constant, indicating an endogenous regulated process (FIGS. 24C and 24D). Using 13C-glucose, 13C-glutamine, and 13C-palmitate as tracers, it was found that 60% of the acetate that is released into the media is derived from glucose and almost none from fatty acids or glutamine, an otherwise major contributor to TCA intermediates in ECs13 (FIG. 24E), suggesting that labeled acetate does not originate indiscriminately from mitochondrial Ac-CoA pools but specifically from glucose. Equally surprisingly, CRISPR deletion of ACLY in ECs in vitro had no impact on acetate secretion, indicating that acetate production by ECs was entirely independent of ACLY (FIG. 24F), despite being generated by glucose, a pathway that typically requires ACLY. These findings mirror the lack of ACLY contribution to Ac-CoA levels or EndMT.

To further probe the link between acetate and Ac-COA, the same ¹³C tracers were used to measure acetate incorporation into the Ac-CoA pool. It was found that acetate indeed is a prominent contributor to the Ac-CoA pool in ECs, accounting for a striking 30%-50% of Ac-COA carbons in the acyl group, depending on ambient acetate concentrations (plasma acetate concentrations range from 100 to 500 mM) (FIGS. 24G and 24H). The vast majority of this acetate-derived Ac-COA pool is cytoplasmic because nearly no contribution by ¹³C-acetate to succinyl-CoA was observed (FIG. 24I) (glucose and acetate provide carbons to succinyl-CoA via mitochondrial Ac-COA, whereas glutamine bypasses Ac-CoA). Correspondingly, ¹³C-palmitate labeling accounted for only a small percent of Ac-COA labeling suggesting that in ECs the mitochondrial Ac-COA pool is smaller than the cytoplasmic pool. Furthermore, it was found that the incorporation of acetate into Ac-COA was largely dependent on ACSS2 (FIG. 24J) but not ACLY (FIG. 24K). In agreement with these data, treatment of HUAECs with TGFβ resulted in increased accumulation of glucose-derived acetate in the cell culture media (FIG. 24L).

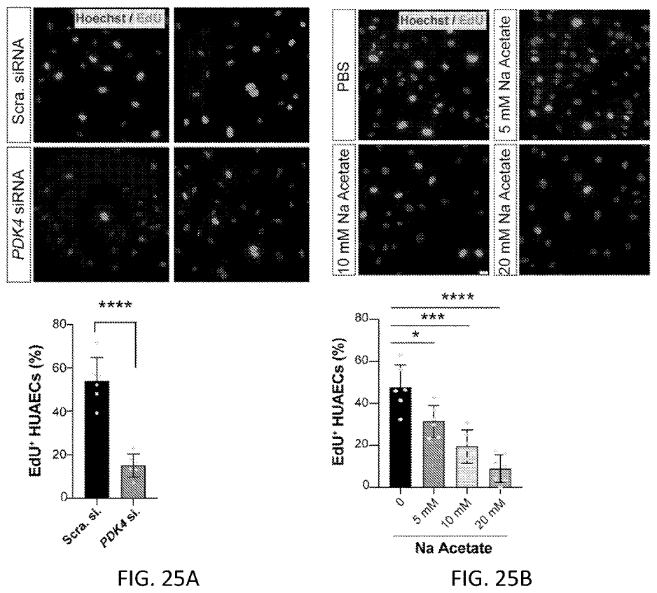

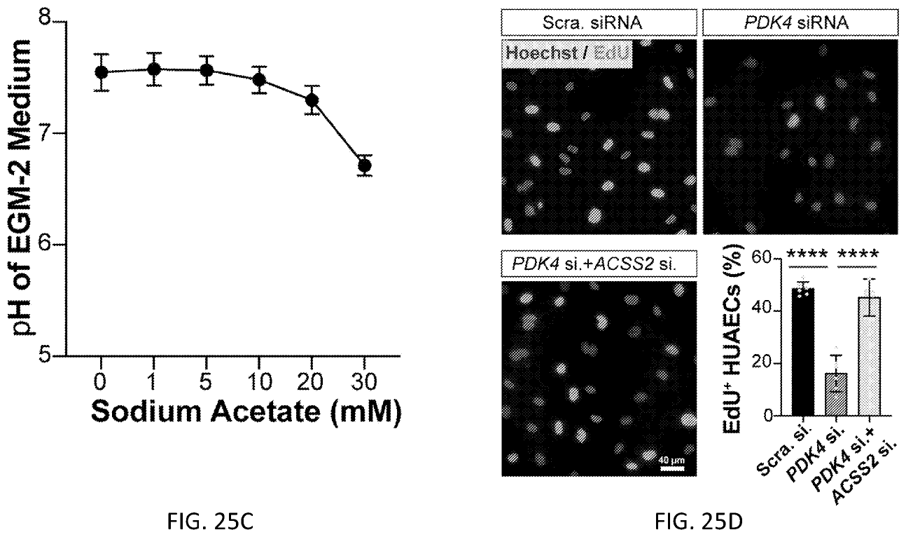

These results are consistent with a pathway whereby TGFβ stimulates glucose uptake and glucose-derived production of acetate, in an atypical ACLY-independent fashion, which is then converted to Ac-COA in the cytoplasm by ACSS2 to drive EndMT. To directly test this implication of acetate as a central modulator of EndMT, studies next examined whether increasing acetate concentration in the media would, by itself, induce EndMT. Five days of supplementation of cultured HUAECs with acetate induced a dose-dependent increase in expression of EndMT markers (FIG. 8A), a change in morphology (FIG. 8B), and an increased shift of P-SMAD2/3 to the nucleus (FIG. 8C), all consistent with induction of TGFβ signaling and EndMT. Edu labeling was used to test whether proliferation of ECs was suppressed by decreased PDK4 expression or sodium acetate treatment. A significant reduction in endothelial proliferation was also observed, measured by Edu labeling, after PDK4 knockdown (FIG. 25A), and a dose-dependent decrease after treatment with sodium acetate (FIG. 25B). Of note, sodium acetate treatment in concentrations below 20 mM had no effect on tissue culture medium pH (FIG. 25C). Furthermore, ACCS2 knockdown in cells subjected to PDK4 siRNA treatment restored normal EC morphology (FIG. 10K). Expression of mesenchymal markers and the inhibition of cell proliferative activity induced by PDK4 KD were also rescued by knocking down ACSS2 expression (FIGS. 10L and 25D). These data place acetate squarely at the heart of Ac-COA biology in ECs and reveal a picture whereby acetate is robustly produced by ECs, in an atypical fashion independent of ACLY, and substantially contributes to the Ac-CoA pool, in an ACCS2-dependent fashion.

### Example 8: ACSS2-Generated Ac-COA Regulates TGFβ Signaling

Studies next examined how an increase in the cytoplasmic Ac-CoA level induces EndMT. Because TGFβ signaling is the main driver of EndMT, changes in the cytosolic and nuclear SMADs content was first observed. As expected, TGFβ stimulation resulted in a shift of R-SMADs 2 and 3 and the common SMAD4 from the cytoplasm to the nucleus (FIGS. 26A and 26B). Importantly, the nuclear shift of SMADs 2 and 4 (but not that of SMAD3) was strongly reduced by ACSS2 knockdown, whereas ACLY knockdown had no effect (FIGS. 26A-26C). Furthermore, phosphorylation of R-SMADs induced by TGFβ treatment was almost completely attenuated in the presence of ACSS2 knockdown (FIGS. 27A and 27B). In addition, although TGFβ treatment of HUAECs led to a robust 3.4-fold increase in expression of the inhibitory SMAD7, there was only a minor (~1.4- to 1.6-fold) increase in SMAD7 acetylation (FIG. 27). Finally, even in the absence of TGFβ treatment, acetylation of SMAD2 and 4 (but not SMAD3) was diminished after ACSS2 knockdown (FIG. 26D). TGFβ treatment of HUAECs induced a further increase in acetylation of R-SMADs that was reduced by ACSS2 knockdown (FIG. 27D). Consistent with these TGFβ treatment results, both SMAD2 acetylation and the nuclear shift of SMAD2/3 were enhanced after adenoviral expression of ACSS2 in HUAECs (FIGS. 27E and 27F).

In addition to affecting SMAD2 and SMAD4 acetylation and nuclear shift, TGFβ stimulation also led to a significant increase in ALK5 expression that was reversed by ACSS2 KD (FIGS. 26E and 26F). Because ALK5 expression is known to be TGFβ-dependent, ALK5 levels after SMAD2 or SMAD4 knockdowns were assessed and observed a significant decrease in its expression (FIGS. 26G and 26H).

These results show that a TGFβ-induced increase in Ac-COA levels is leading to increased acetylation of SMADs and ALK5 and increased ALK5 expression. To further link these changes in ALK5 expression to acetylation, a tagged ALK5 construct was expressed in HUAECs. Increasing ACSS2 levels, using an adenoviral-mediated ACSS2 transduction, resulted in increased ALK5 expression (FIG. 26I), whereas ACSS2 KD led to a decrease in ALK5 expression (FIG. 26J). Finally, a PDK4 knockdown that mimics the effect of TGFβ treatment also resulted in increased ALK5 expression and increased phosphorylation of R-SMADs (FIGS. 26K and 27G-27I). Treatment with the PDK inhibitor DCA had a similar effect (FIG. 27J).

Taken together, these data show that ACSS2-driven Ac-CoA production leads to ALK5 acetylation, thereby increasing its expression. To test this directly, blotting was carried out using an anti-Ac-lysine antibody of TGFβ-treated HUAECs transduced with a tagged ALK5 construct. In agreement with above data, TGFβ treatment resulted in increased ALK5 acetylation (FIG. 28A) as did a PDK4 knockdown (FIG. 28B), whereas ACSS2 knockdown strongly reduced it (FIG. 28C).

To determine how acetylation affects ALK5 expression, ALK5 protein half-life in HUAECs and human umbilical vein ECs (HUVECs) was measured+. There was a significant increase in ALK5 protein stability after acetylation-inducing PDK4 knockdown (FIG. 28D), whereas ACSS2 knockdown had the opposite effect (FIGS. 28E and 27K). Next, HUAECs and HUVECs expressing a tagged ALK5 were treated with a proteasome inhibitor MG132 and lysosome inhibitor chloroquine. Treatment with the former, but not the latter, increased ALK5 levels after ACSS2 knockdown, suggesting that deacetylated ALK5 undergoes rapid proteasome-dependent degradation (FIGS. 28F, 28G, 27L, and 27M). Together, these data show that ALK5 acetylation results in increased protein half-life due to decreased proteasomal degradation (FIG. 28H).

Studies next set out to identify the ALK5 acetylation sites. Expression of a human ALK5 protein fused with the Flag tag in 293T cells led to detection of strongly acetylated ALK5 after immunoprecipitation with an anti-Flag antibody (FIG. 29A). LC-MS/MS analysis of the fusion construct pointed to Lys490 as the likely acetylation residue in the ALK5 protein (FIGS. 29B and 29C). However, transfection of 293T cells with either wild-type ALK5 or an ALK5 carrying a K490R mutation (lysine [K] to arginine [R] substitutions at the Lys490 site) did not show any decrease in ALK5 acetylation, implying that other site(s) were responsible (FIG. 29D). Follow-up studies therefore separately mutated to arginine each one of the 25 lysine residues in the human ALK5 protein creating 25 different ALK5 mutant-Flag constructs. Every mutant construct was then transfected into 293T cells, and the extent of acetylation was assessed after immunoprecipitation with an anti-Flag antibody. A strong reduction in acetylation of ALK5 proteins carrying K223R, K343R, K391R, and K449R mutations was observed (FIG. 29E). Therefore, these five lysine residues (K223, K343, K391, K449, and K490) are the principal acetylation sites in ALK5.

### Example 9: ACSS2 Regulates EndMT In Vivo

To assess if endothelial ACSS2 plays a similar role in endothelial metabolism in vivo, a series of studies next investigated its role in the development and progression of atherosclerosis, an End-MT-driven disease. Immunocytochemical analysis of endothelial ACSS2 expression in ascending aortas of 13 normal organ donors with either minimal (n=7) or mild/moderate (n=6) extent of atherosclerosis revealed a significant increase in ACSS2 expression (FIGS. 30A and 30B). A similar analysis of endothelial ACSS2 expression in the severely atherosclerotic brachiocephalic trunk and aortic root from Apoe−/− mice after 3 months of high-fat diet revealed substantial ACSS2 expression in the endothelium (FIGS. 30C and 30D).

Given that in vitro and in vivo data are pointing to likely ACSS2 involvement in the development and progression of atherosclerosis, studies evaluated whether suppression of endothelial ACSS2 expression would influence the development of atherosclerosis. To this end, mice with an inducible EC-specific deletion of Acss2 on the ApoE null background were generated (Cdh5CreERT2; Accs2fl/fl;Apoe−/− thereafter ACSS2iECKO;ApoE) (FIGS. 31A and 31B). Activation of the Cdh5Cre by tamoxifen in mice of 5-6 weeks of age resulted in a high-efficiency deletion of the endothelial Acss2 gene (FIG. 31D). Two weeks later, ~8-week-old ApoE−/− control mice and ACSS2iECKO;ApoE mice were placed on a high-fat diet (n=10 each, FIG. 31C) and then sacrificed 3 months later. The whole aorta oil red O staining was used to assess the total atherosclerotic burden. Compared with Apoe−/− mice, ACSS2iECKO;ApoE mice showed a highly significant reduction in atherosclerosis extent uniformly along the length of the entire aorta (FIGS. 32A, 32B, 31E, and 31F). Histologic analysis of aortic root sections demonstrated a significant reduction of plaque area in ACSS2iECKO;ApoE mice (FIG. 32C). Analysis of the total cholesterol and triglycerides levels in the plasma showed no differences between ACSS2iECKO;ApoE and ApoE−/− mice demonstrating that the plasma lipids are not affected by the EC-specific deletion of Acss2 (FIGS. 32D, 32E, 31G, and 31H). Finally, in agreement with the overall reduction of the atherosclerotic burden and atherosclerotic plaque sizes, immunostaining of the aortic root sections demonstrated a decrease in fibronectin and collagen-1 deposition, reduced VCAMI expression and reduces number of blood-derived onocytes/macrophages (CD68) in ACSS2iECKO;ApoE mice (FIGS. 32F-32I).

To evaluate ACSS2 as a potential therapeutic target in atherosclerosis, nanoparticle-delivered ACSS2 siRNA was used to knockdown endothelial ACSS2 expression. To this end, 2 months old ApoE−/− mice were placed on high-fat diet and assigned to every 10-day injections of 7C1 nanoparticles loaded with either unmodified or modified ACSS2 siRNA (n=9 each) or no treatment controls (n=12, FIGS. 33A and 33B). The mice were sacrificed 3 months later and whole aorta oil red O staining was used to assess total atherosclerotic burden. Both ACSS2 siRNA-treated groups showed a highly significant reduction in atherosclerosis extent in agreement with ACSS2 knockout data (FIGS. 33C and 33D).

### Example 10: ACSS2 and Inhibition of PDK4 Drive Positive Feedback Loop

Together, our data unveil a linear pathway whereby TGFβ induces glucose conversion to acetate, followed by conversion of acetate to cytosolic Ac-COA by ACSS2, ultimately leading to increased expression of SMADs and ALK5 and persistent activation of the TGFβ signaling pathway. However, TGFβ signaling was also observed to suppress PDK4, thus setting up a positive-feedback loop (FIG. 32J). Further substantiating the existence of this positive-feedback loop, it was found that ACSS2 knockdown partially re-induced PDK4 (i.e., antagonized the siRNA effect), thereby reversing PDH complex activation (i.e., dephosphorylation) induced by PDK4 knockdown (FIG. 34A). Consequently, ACSS2 knockdown also reduced EndMT markers and restoration of EC-specific gene expression induced by PDK4 siRNA (FIGS. 34B and 34C). Conversely, in the context of pro-EndMT TGFβ signaling, knockdown of PDK4 partially reversed the siACSS2-mediated block of EndMT induction by TGFβ (FIGS. 34D-S8G). Thus, ACSS2 acts both upstream of PDK4, via generation of Ac-COA and promotion of SMAD2,3 binding to PDK4 promoter that leads to downregulation of PDK4 expression, and downstream of PDK4, via induced production of Ac-COA, establishing a positive-feedback loop that drives EndMT as manifested in vivo by the development of atherosclerosis (FIG. 32J).

### ENUMERATED EMBODIMENTS

The following enumerated embodiments are provided, the numbering of which is not to be construed as designating levels of importance.

Embodiment 1 provides a method of inhibiting endothelial-mesenchymal transition (EndMT) in a cell comprising contacting the cell with an effective amount of an inhibitor of Acetyl COA Synthetase Short chain family member 2 (ACSS2).

Embodiment 2 provides the method of claim 1, wherein the EndMT occurs as a result of chronic vascular inflammation.

Embodiment 3 provides the method of claim 1, wherein the inhibition of ACSS2 opposes pathogenic Transforming Growth Factor-Beta (TGFβ) in the cell.

Embodiment 4 provides the method of claim 1, wherein inhibition of EndMT is used to treat a disease associated with chronic vascular inflammation.

Embodiment 5 provides the method of claim 4, wherein the disease is selected from the group consisting of atherosclerosis, arteriosclerosis, pulmonary hypertension, myocardial infarction, and stroke.

Embodiment 6 provides the method of claim 1, wherein the inhibitor of ACSS2 is an inhibitory RNA which reduces the expression of ACSS2.

Embodiment 7 provides the method of claim 6, wherein the inhibitory RNA is an siRNA.

Embodiment 8 provides the method of claim 7, wherein the siRNA is encoded by a nucleic acid sequence set forth in SEQ ID NOs: 1-8.

Embodiment 9 provides a pharmaceutical composition comprising
a. an inhibitor of ACSS2,
b. a lipid nanoparticle, and
c. a pharmaceutically acceptable carrier.

Embodiment 10 provides the pharmaceutical composition of claim 9, wherein the inhibitor of ACSS2 is an inhibitory RNA which reduces the expression of ACSS2.

Embodiment 11 provides the pharmaceutical composition of claim 10, wherein the inhibitory RNA is an siRNA.

Embodiment 12 provides the pharmaceutical composition of claim 11, wherein the siRNA is encoded by the nucleotide sequence set forth in SEQ ID NO:s 1-8.

Embodiment 13 provides a method of treating a vascular inflammatory disease in a subject in need thereof, the method comprising administering an effective amount of an inhibitor of Acetyl CoA Synthetase Short chain family member 2 (ACSS2).

Embodiment 14 provides the method of 13, wherein the vascular inflammatory disease is a chronic vascular inflammation disease

Embodiment 15 provides the method of claim 14, wherein the chronic vascular inflammatory disease is selected from the group consisting of atherosclerosis, myocardial infarction, pulmonary hypertension, and stroke.

Embodiment 16 provides the method of claim 13, wherein the vascular inflammatory disease results from a vascular graft failure.

Embodiment 17 provides the method of claim 13, wherein the vascular inflammatory disease is associated with a fibrotic state of the vessels.

Embodiment 18 provides the method of claim 13, wherein the inhibitor of ACSS2 is an inhibitory RNA that reduces the expression of ACSS2.

Embodiment 19 provides the method of claim 18, wherein the inhibitory RNA is an siRNA.

Embodiment 20 provides the method of claim 19, wherein the siRNA is encoded by the nucleic acid sequence set forth in SEQ ID NOs. 1-8.

Embodiment 21 provides the method of claim 13, wherein the ACSS2 inhibitor is the pharmaceutical composition of any one of claims 11-15.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of inhibiting endothelial-mesenchymal transition (EndMT) in a cell comprising contacting the cell with an effective amount of an inhibitor of Acetyl CoA Synthetase Short chain family member 2 (ACSS2).

2. The method of claim 1, wherein the EndMT occurs as a result of chronic vascular inflammation.

3. The method of claim 1, wherein the inhibition of ACSS2 opposes pathogenic Transforming Growth Factor-Beta (TGFβ) in the cell.

4. The method of claim 1, wherein inhibition of EndMT is used to treat a disease associated with chronic vascular inflammation.

5. The method of claim 4, wherein the disease is selected from the group consisting of atherosclerosis, arteriosclerosis, pulmonary hypertension, myocardial infarction, and stroke.

6. The method of claim 1, wherein the inhibitor of ACSS2 is an inhibitory RNA which reduces the expression of ACSS2.

7. The method of claim 6, wherein the inhibitory RNA is an siRNA.

8. The method of claim 7, wherein the siRNA comprises one or more of SEQ ID NOs: 1-8.

9. A pharmaceutical composition comprising
a. an inhibitor of ACSS2,
b. a lipid nanoparticle, and
c. a pharmaceutically acceptable carrier.

10. The pharmaceutical composition of claim 9, wherein the inhibitor of ACSS2 is an inhibitory RNA which reduces the expression of ACSS2.

11. The pharmaceutical composition of claim 10, wherein the inhibitory RNA is an siRNA.

12. The pharmaceutical composition of claim 11, wherein the siRNA comprises one or more of SEQ ID NOs: 1-8.

13. A method of treating a vascular inflammatory disease in a subject in need thereof, the method comprising administering an effective amount of an inhibitor of Acetyl COA Synthetase Short chain family member 2 (ACSS2).

14. The method of 13, wherein the vascular inflammatory disease is a chronic vascular inflammation disease

15. The method of claim 14, wherein the chronic vascular inflammatory disease is selected from the group consisting of atherosclerosis, myocardial infarction, pulmonary hypertension, and stroke.

16. The method of claim 13, wherein the vascular inflammatory disease results from a vascular graft failure.

17. The method of claim 13, wherein the vascular inflammatory disease is associated with a fibrotic state of the vessels.

18. The method of claim 13, wherein the inhibitor of ACSS2 is an inhibitory RNA that reduces the expression of ACSS2.

19. The method of claim 18, wherein the inhibitory RNA is an siRNA.

20. The method of claim 19, wherein the siRNA comprises one or more of SEQ ID NOs: 1-8.

21. (canceled)
